(19) United States
(12) Patent Application Publication
(10) Pub. No.: US 2025/0164435 A1
(43) Pub. Date: May 22, 2025

(54) SENSOR ELEMENT AND GAS SENSOR

(71) Applicant: NGK INSULATORS, LTD., Nagoya-shi (JP)

(72) Inventors: Yuki KAJITA, Kasugai-shi (JP); Takamasa MATSUBARA, Komaki-shi (JP)

(21) Appl. No.: 18/953,676

(22) Filed: Nov. 20, 2024

(30) Foreign Application Priority Data
Nov. 22, 2023 (JP) 2023-198185

(51) Int. Cl. G01N 27/407; G01N 33/00

(52) U.S. Cl. CPC G01N 27/4071; G01N 27/4075; G01N 27/4078; G01N 33/0027

(57) ABSTRACT

A sensor element configured to detect a concentration of a specific gas in a measurement-object gas, the sensor element includes an element body having a columnar shape elongate in a longitudinal direction, the element body having a front end and a rear end that are opposite ends in the longitudinal direction and further having a peripheral face that is a surface extending in the longitudinal direction, an inner electrode disposed inside the element body; and a conducting section including an inner conducting section and an outer conducting section, the outer conducting section being electrically continuous with the inner conducting section, wherein a part of the outer conducting section that covers the inner conducting section and/or at least a part of the inner conducting section is a dense segment that has a dense composition with precious metal, alumina, and silica.

### CROSS-REFERENCE TO RELATED APPLICATION

The present application claims priority from Japanese application JP2023-198185, filed on Nov. 22, 2023, the content of which is hereby incorporated by reference into this application.

### BACKGROUND OF THE INVENTION

### 1. Field of the Invention

The present invention relates to a sensor element and a gas sensor.

### 2. Description of the Related Art

A known gas sensor in the related art includes a sensor element configured to detect the concentration of a specific gas, such as NOx, in a measurement-object gas, such as exhaust gas from an internal combustion engine (see PTL 1, for example). The sensor element disclosed in PTL 1 includes an element body, an inner electrode, a terminal, and a lead. The element body includes an oxygen-ion-conductive solid electrolyte layer and has a columnar shape elongate in a longitudinal direction. The element body has a front end and a rear end that are opposite ends in the longitudinal direction, and a peripheral face that is a surface extending in the longitudinal direction. A part of the element body that is at the front end is allowed to be exposed to a measurement-object gas. The inner electrode is disposed inside the element body. The terminal is disposed at a part of the peripheral face of the element body that is near the rear end. The lead is disposed in such a manner as to connect the inner electrode and the terminal to each other. The lead includes an inner section disposed inside the element body, and a peripheral-face conducting section disposed on the peripheral face and exposed from the element body. The terminal and the peripheral-face conducting section are each composed of an electrically conductive material obtained by mixing noble metal powder and alumina powder. Thus, the adhesive strength of the terminal and the peripheral-face conducting section with respect to the solid electrolyte layer is increased.

### CITATION LIST

### Patent Literature

PTL 1: JP 4628920 B

### SUMMARY OF THE INVENTION

In such a sensor element, some kind of gas outside the sensor element (element body) may move through the inside of the element body, along the inner section of the lead or a gap between the inner section of the lead and the element body, and reach the inner electrode. If such a gas reaches the inner electrode, the accuracy in the detection of concentration of the specific gas may be deteriorated. Accordingly, for example, it is conceivable that a dense peripheral-face conducting section may be provided over the inner section of the lead to make the gas less likely to reach the inner electrode. In the sensor element disclosed in PTL 1, the peripheral-face conducting section contains alumina. Therefore, the adhesive strength between the peripheral-face conducting section and the element body is increased. Instead, the porosity of the peripheral-face conducting section tends to be increased, making it difficult to obtain a dense peripheral-face conducting section.

The present invention has been made to solve the above problems, and a main object thereof is to increase both denseness and adhesiveness in a part of the conducting section that contributes to the less likeliness of the gas reaching the inner electrode.

To achieve the above main object, the present invention employs the following solutions.

[1] A sensor element according to the present invention is a sensor element configured to detect a concentration of a specific gas in a measurement-object gas, the sensor element comprising: an element body including an oxygen-ion-conductive solid electrolyte layer and having a columnar shape elongate in a longitudinal direction, the element body having a front end and a rear end that are opposite ends in the longitudinal direction and further having a peripheral face that is a surface extending in the longitudinal direction, a part of the element body that is at the front end being allowed to be exposed to the measurement-object gas; an inner electrode disposed inside the element body; and a conducting section including an inner conducting section and an outer conducting section, the inner conducting section being disposed inside the element body and being electrically continuous with the inner electrode, the outer conducting section including a connector electrode disposed at a part of the peripheral face that is near the rear end, the outer conducting section further including a segment disposed on the peripheral face and/or a segment disposed on the peripheral face and exposed to an outside of the sensor element, the outer conducting section being electrically continuous with the inner conducting section, wherein a part of the outer conducting section that covers the inner conducting section and/or at least a part of the inner conducting section is a dense segment that has a dense composition with noble metal, alumina, and silica.

In this sensor element, the dense segment is the part of the outer conducting section that covers the inner conducting section and/or at least the part of the inner conducting section, and the dense segment contains not only noble metal but also alumina and silica. Therefore, the dense segment has increased adhesiveness. Furthermore, since the dense segment contains silica, the dense segment has increased denseness. Therefore, both denseness and adhesiveness are increased in the part of the outer conducting section that covers the inner conducting section and or at least the part of the inner conducting section, i.e., the dense segment included in the conducting section and that contributes to the less likeliness of the gas reaching the inner electrode. The present inventors have demonstrated such facts through experiments, analyses, and the like.

[2] In the sensor element according to the present invention (the sensor element according to [1] above), letting volumes of alumina and silica contained in the dense segment be Va [vol %] and Vs [vol %], respectively, the dense segment may satisfy Va/Vs≤1.5. Thus, the denseness of the dense segment is further increased.

[3] In the sensor element according to the present invention (the sensor element according to [1] or [2] above), letting volumes of noble metal, alumina, and silica contained in the dense segment be Vp [vol %], Va [vol %], and Vs [vol %], respectively, the dense segment may satisfy 1.5≤Vp/(Va+Vs). Thus, a more assured effect of increasing the denseness of the dense segment is obtained.

[4] In the sensor element according to the present invention (the sensor element according to [3] above), the dense segment may satisfy 2.6≤Vp/(Va+Vs). Thus, the denseness of the dense segment is further increased.

[5] In the sensor element according to the present invention (the sensor element according to any of [1] to [4] above), a ratio of a total mass of alkali metal and alkaline-earth metal contained in the dense segment on an oxide basis may satisfy 0.1 wt % or smaller. Such a composition lowers the probability of reduction in the conductivity of the dense segment that may be caused by the electromigration of the alkali metal and/or alkaline-earth metal in the dense segment, and thus lowers the probability of accuracy deterioration in the detection of concentration of the specific gas that may be caused by the reduction in the conductivity.

[6] In the sensor element according to the present invention (the sensor element according to any of [1] to [5] above), the inner electrode may be a measurement electrode to be used in detecting the concentration of the specific gas.

[7] In the sensor element according to the present invention (the sensor element according to any of [1] to [6] above), the element body may be a layered body obtained by stacking a plurality of layers including the solid electrolyte layer in a layered direction that is orthogonal to the longitudinal direction, the layered body may have a first face and a second face that are opposite end faces in the layered direction; and a third face and a fourth face that are opposite end faces in a direction orthogonal to the longitudinal direction and to the layered direction, the first to fourth faces each being the peripheral face, the inner conducting section may include an inner lead that is routed to the third face or the fourth face, and the part of the outer conducting section that covers the inner conducting section may be a peripheral-face lead disposed on the third face or the fourth face and covering the inner lead.

[8] In the sensor element according to the present invention (the sensor element according to any of [1] to [6] above), the element body may be a layered body obtained by stacking a plurality of layers including the solid electrolyte layer in a layered direction that is orthogonal to the longitudinal direction, the layered body may have a first face and a second face that are opposite end faces in the layered direction and are each the peripheral face, the element body may have a through-hole with an opening provided in a part of the peripheral face that is near the rear end, the peripheral face being the first face or the second face, the through-hole extending through one or more of the plurality of layers in the layered direction, the outer conducting section may be the connector electrode, and the inner conducting section may include a through-hole conductor disposed in the through-hole and covered by the connector electrode.

[9] In the sensor element according to the present invention (the sensor element according to any of [1] to [6] above), the element body may be a layered body obtained by stacking a plurality of layers including the solid electrolyte layer in a layered direction that is orthogonal to the longitudinal direction, the layered body may have a first face and a second face that are opposite end faces in the layered direction and are each the peripheral face, the element body may have a through-hole with an opening provided in a part of the peripheral face that is near the rear end, the peripheral face being the first face or the second face, the through-hole extending through one or more of the plurality of layers in the layered direction, the inner conducting section may include a through-hole conductor disposed in the through-hole and exposed to the outside at the opening, and the dense segment may include at least a part of the through-hole conductor.

[10] The sensor element according to the present invention (the sensor element according to any of [1] to [9] above) may be a sensor element to be included in a gas sensor, the gas sensor including the sensor element; a case having a cylindrical shape elongate in the longitudinal direction of the sensor element, the case having a second front end and a second rear end that are opposite ends in the longitudinal direction, the sensor element being disposed inside the case; and a sealing member sealing the second rear end of the case.

[11] A gas sensor according to the present invention includes the sensor element according to any of [1] to [9] above; a case having a cylindrical shape elongate in the longitudinal direction of the sensor element, the case having a second front end and a second rear end that are opposite ends in the longitudinal direction, the case housing the sensor element; and a sealing member sealing the second rear end of the case.

Since this gas sensor includes the above sensor element, the gas sensor produces the same advantageous effects as those produced by the above sensor element: for example, an advantageous effect of increasing both denseness and adhesiveness in the part of the conducting section that contributes to the less likeliness of the gas reaching the inner electrode.

### BRIEF DESCRIPTION OF THE DRAWINGS

FIG. 1 is a vertical sectional view of a gas sensor 100.

FIG. 2 is a schematic sectional view of a sensor element 101, illustrating an exemplary configuration thereof.

FIG. 3 is a block diagram illustrating an electrical connection relationship between a control device 95 and relevant elements including cells.

FIG. 4 is a perspective view of a rear end part of an element body 102.

FIG. 5 is an enlarged sectional view of a part including a conducting section 74 provided for a measurement electrode 44.

FIG. 6 is a sectional view of a part of a sensor element 201 according to a modification.

FIG. 7 is a sectional view of a part of a sensor element 301 according to another modification.

FIG. 8 is a sectional view of a part of a sensor element 401 according to yet another modification.

FIG. 9 is a sectional view of a part of a sensor element 501 according to yet another modification.

FIG. 10 is a schematic sectional view of a sensor element 601 according to yet another modification.

### DETAILED DESCRIPTION OF THE INVENTION

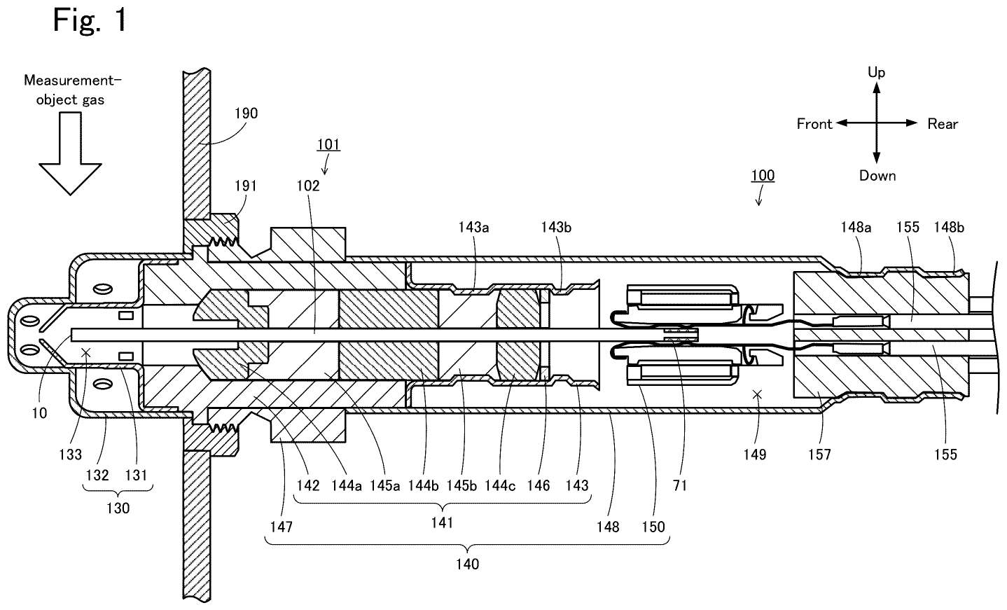

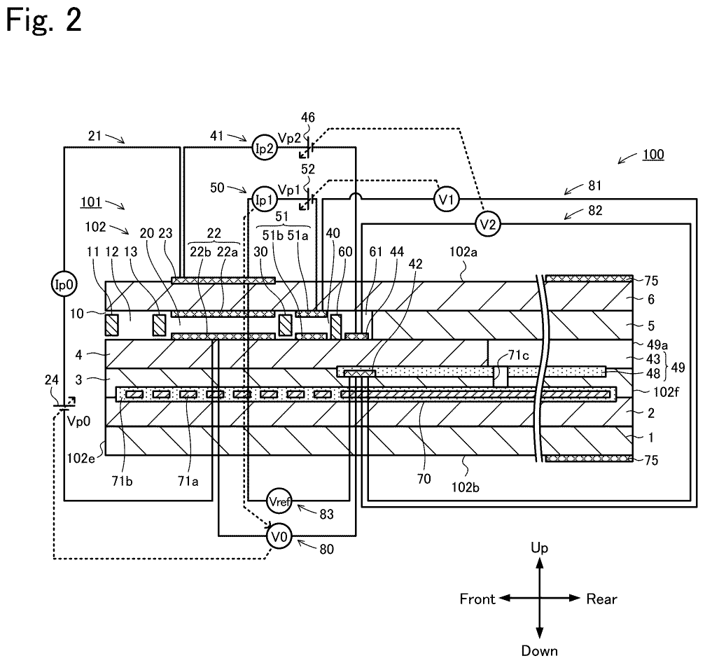

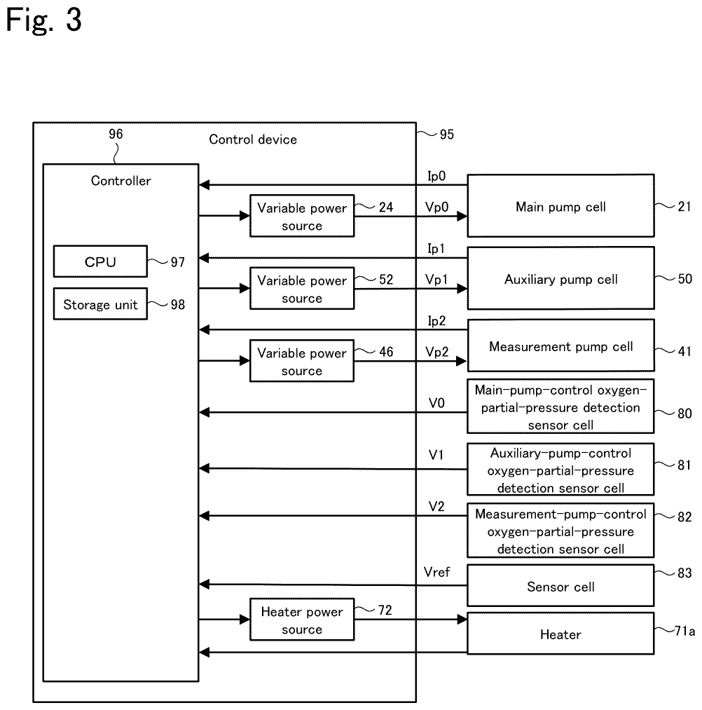

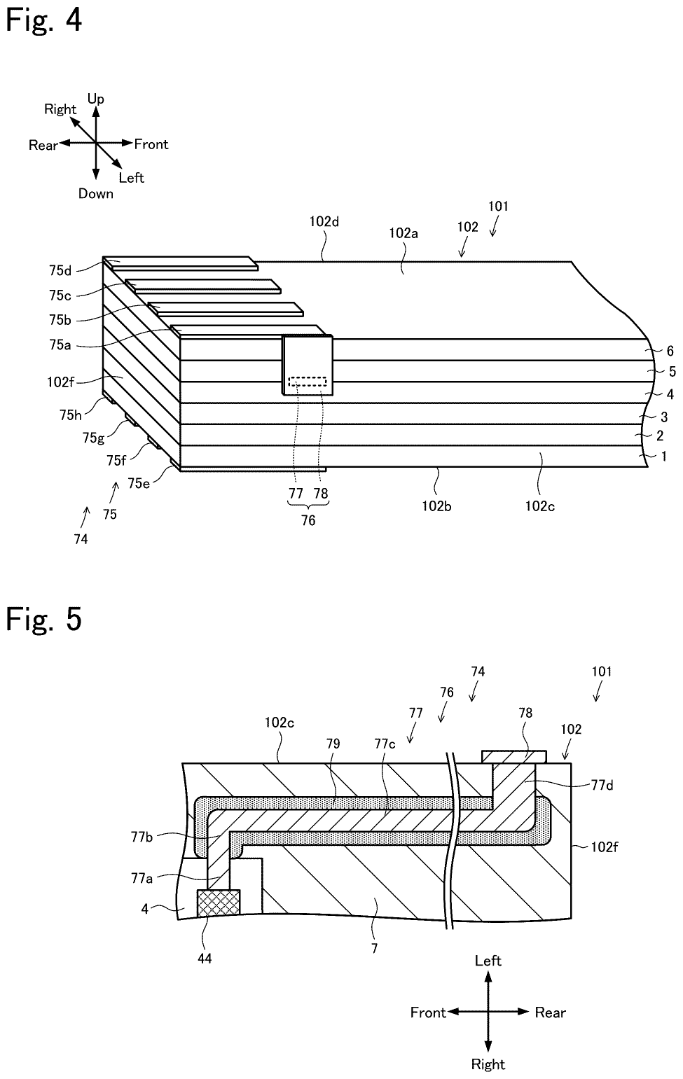

Embodiments of the present invention will now be described with reference to the drawings. FIG. 1 is a vertical sectional view of a gas sensor 100 according to an embodiment of the present invention. FIG. 2 is a schematic sectional view of a sensor element 101 included in the gas sensor 100, illustrating an exemplary configuration thereof. FIG. 3 is a block diagram illustrating an electrical connection relationship between a control device 95 and relevant elements of the sensor element 101, including cells and a heater 71a. FIG. 4 is a perspective view of a rear end part of an element body 102 included in the sensor element 101. FIG. 5 is an enlarged sectional view of a part of the sensor element 101, illustrating a measurement electrode 44 and a conducting section 74 provided therefor. The element body 102 of the sensor element 101 has an elongate rectangular parallelepiped shape. The longitudinal direction of the element body 102 (the left-right direction in FIG. 2) is defined as the front-rear direction. The thickness direction of the element body 102 (the up-down direction in FIG. 2) is defined as the up-down direction. The width direction of the element body 102 (the direction perpendicular to the front-rear direction and to the up-down direction) is defined as the left-right direction. Since the element body 102 is a rectangular parallelepiped body, as illustrated in FIGS. 2, 4, and 5, the element body 102 has the following six faces forming the outer surfaces of solid electrolyte layers constituting the element body 102: a first face 102a (upper face), a second face 102b (lower face), a third face 102c (left face), a fourth face 102d (right face), a fifth face 102e (front end face), and a sixth face 102f (rear end face).

As illustrated in FIG. 1, the gas sensor 100 includes the sensor element 101 having the element body 102, a protection cover 130 protecting a part of the element body 102 that is at the front end, and a sensor assembly 140 including a connector 150 electrically continuous with the sensor element 101. As illustrated in the drawing, the gas sensor 100 is attached to a pipe 190, such as an exhaust pipe of an internal combustion engine (diesel engine, gasoline engine, or the like) included in a vehicle. Exhaust gas from the internal combustion engine is taken as a measurement-object gas. The gas sensor 100 is used to measure (detect) specific gas concentration, which refers to the concentration of a specific gas, such as NOx; 02; or ammonia, contained in the measurement-object gas. In the present embodiment, the specific gas concentration to be measured by the gas sensor 100 is NOx concentration.

The protection cover 130 includes a bottomed cylindrical inner protection cover 131 covering the front end part of the element body 102, and a bottomed cylindrical outer protection cover 132 covering the inner protection cover 131. The inner protection cover 131 and the outer protection cover 132 each have a plurality of holes for allowing the measurement-object gas to flow into the protection cover 130. The space enclosed by the inner protection cover 131 serves as a sensor element chamber 133. The front end part of the element body 102 is positioned in the sensor element chamber 133.

The sensor assembly 140 includes an element sealing unit 141 that seals and secures the sensor element 101, a bolt 147 and an external cylinder 148 that are attached to the element sealing unit 141, and the connector 150 that is in contact with and electrically connected to connector electrodes 75 disposed on surfaces (upper and lower surfaces) of the element body 102 of the sensor element 101 at a rear end part.

The element sealing unit 141 includes a cylindrical main metal fitting 142, and a cylindrical inner cylinder 143 welded coaxially to the main metal fitting 142. The element sealing unit 141 further includes ceramic supporters 144a to 144c, compacts 145a and 145b, and a metal ring 146 that are sealed in a through-hole provided on the inner side of the main metal fitting 142 and the inner cylinder 143. The sensor element 101 is located on the center axis of the element sealing unit 141 and extends through the element sealing unit 141 in the front-rear direction. The inner cylinder 143 has a diameter reduction section 143a for pressing the compact 145b toward the center axis of the inner cylinder 143, and a diameter reduction section 143b for pressing the ceramic supporters 144a to 144c and the compacts 145a and 145b frontward via the metal ring 146. The pressing forces generated by the diameter reduction sections 143a and 143b compress the compacts 145a and 145b between the sensor element 101 and the combination of the main metal fitting 142 and the inner cylinder 143. Thus, the compacts 145a and 145b seal between the sensor element chamber 133 in the protection cover 130 and a space 149 in the external cylinder 148, and also secure the sensor element 101.

The bolt 147 is secured coaxially to the main metal fitting 142 and has a male threaded section around the outer peripheral surface thereof. The male threaded section of the bolt 147 is screwed into a securing member 191 welded to the pipe 190 and having a female threaded section around the inner peripheral surface thereof. Thus, the gas sensor 100 is secured to the pipe 190, with a part of the gas sensor 100 inclusive of the front end part of the element body 102 of the sensor element 101 and the protection cover 130 protruding into the pipe 190.

The external cylinder 148 encloses the inner cylinder 143, the sensor element 101, and the connector 150. A plurality of lead wires 155 are connected to the connector 150 and are routed to the outside from the rear end of the external cylinder 148. The lead wires 155 are electrically continuous with electrodes (to be described below) of the sensor element 101 via the connector 150. At the rear end of the external cylinder 148, the gap between the external cylinder 148 and the lead wires 155 is sealed by a rubber stopper 157. The external cylinder 148 has swaged sections 148a and 148b that restrict the front-rear movement of the rubber stopper 157 relative to the external cylinder 148. The space 149 in the external cylinder 148 is filled with a reference gas. The rear end of the element body 102 of the sensor element 101 is positioned in the space 149.

As illustrated in FIG. 2, the sensor element 101 includes the element body 102; cells 21, 41, 50, and 80 to 83; and a heater section 70. The element body 102 is a layered body obtained by stacking six layers in the following order from the lower side in the drawing: a first substrate layer 1, a second substrate layer 2, a third substrate layer 3, a first solid electrolyte layer 4, a spacer layer 5, and a second solid electrolyte layer 6, which are each an oxygen-ion-conductive solid electrolyte layer composed of zirconia (ZrO₂) or the like. The solid electrolyte forming each of these six layers is dense and hermetic. The element body 102 is manufactured by, for example, performing predetermined processing and printing of circuit patterns on ceramic green sheets that are to become the individual layers, subsequently stacking the sheets, and then combining the sheets by performing calcination.

At the front end part of the element body 102 and between the lower surface of the second solid electrolyte layer 6 and the upper surface of the first solid electrolyte layer 4 are provided a gas inlet 10, a first diffusion control section 11, a buffer space 12, a second diffusion control section 13, a first internal cavity (oxygen-concentration adjustment chamber) 20, a third diffusion control section 30, a second internal cavity (oxygen-concentration adjustment chamber) 40, a fourth diffusion control section 60, and a third internal cavity (measurement chamber) 61 that are next to one another and communicate with one another in that order.

The gas inlet 10, the buffer space 12, the first internal cavity 20, the second internal cavity 40, and the third internal cavity 61 are spaces provided inside the sensor element 101 by hollowing out the spacer layer 5, and each have an upper side defined by the lower surface of the second solid electrolyte layer 6, a lower side defined by the upper surface of the first solid electrolyte layer 4, and lateral sides defined by the side surfaces of the spacer layer 5.

The first diffusion control section 11, the second diffusion control section 13, and the third diffusion control section 30 are each provided as two horizontally elongate slits (i.e., openings that are elongate in the direction perpendicular to the plane of the drawing). The fourth diffusion control section 60 is provided as a single horizontally elongate slit (i.e., an opening that is elongate in the direction perpendicular to the plane of the drawing) providing a gap with respect to the lower surface of the second solid electrolyte layer 6. A section extending from the gas inlet 10 to the third internal cavity 61 inclusive is also referred to as a measurement-object gas flow section.

The element body 102 includes a reference-gas introduction portion 49 that allows the reference gas to flow from outside the element body 102 to a reference electrode 42 in the measurement of NOx concentration. The reference-gas introduction portion 49 has a reference-gas introduction space 43 and a reference-gas introduction layer 48. The reference-gas introduction space 43 is a space extending from the sixth face 102f (rear end face) of the element body 102 toward the fifth face 102e (front end face). The reference-gas introduction space 43 is provided at a position between the upper surface of the third substrate layer 3 and the lower surface of the spacer layer 5 and has lateral sides defined by the side surfaces of the first solid electrolyte layer 4. The reference-gas introduction space 43 has an opening at the rear end face of the element body 102. This opening functions as an entrance 49a of the reference-gas introduction portion 49. The reference gas is to be introduced into the reference-gas introduction space 43 through the entrance 49a. The reference-gas introduction portion 49 introduces the reference gas to the reference electrode 42 while applying a predetermined diffusion resistance to the reference gas received through the entrance 49a. In the present embodiment, the reference gas is ambient air.

The reference-gas introduction layer 48 is disposed between the upper surface of the third substrate layer 3 and the lower surface of the first solid electrolyte layer 4. The reference-gas introduction layer 48 is a porous body composed of a ceramic material such as alumina. A part of the upper surface of the reference-gas introduction layer 48 is exposed in the reference-gas introduction space 43. The reference-gas introduction layer 48 is provided over the reference electrode 42. The reference-gas introduction layer 48 allows the reference gas to flow from the reference-gas introduction space 43 to the reference electrode 42.

The reference electrode 42 is interposed between the upper surface of the third substrate layer 3 and the first solid electrolyte layer 4 and is surrounded by the reference-gas introduction layer 48 connected to the reference-gas introduction space 43, as described above. Furthermore, as will be described below, the reference electrode 42 is to be used in measuring the oxygen concentration (oxygen partial pressure) in the first internal cavity 20, the second internal cavity 40, and the third internal cavity 61.

In the measurement-object gas flow section, the gas inlet 10 is open to an external space, so that the measurement-object gas is to be taken into the sensor element 101 from the external space through the gas inlet 10. The first diffusion control section 11 is provided for applying a predetermined diffusion resistance to the measurement-object gas taken in through the gas inlet 10. The buffer space 12 is provided for guiding the measurement-object gas introduced through the first diffusion control section 11 to the second diffusion control section 13. The second diffusion control section 13 is provided for applying a predetermined diffusion resistance to the measurement-object gas to be introduced to the first internal cavity 20 from the buffer space 12. When the measurement-object gas is introduced from outside the sensor element 101 to the first internal cavity 20, the measurement-object gas quickly taken into the sensor element 101 through the gas inlet 10 because of pressure fluctuation of the measurement-object gas (i.e., pulsation of exhaust pressure, if the measurement-object gas is exhaust gas from an internal combustion engine) in the external space is not directly introduced to the first internal cavity 20 but is introduced to the first internal cavity 20 after the pressure fluctuation of the measurement-object gas is negated by traveling through the first diffusion control section 11, the buffer space 12, and the second diffusion control section 13. Accordingly, the pressure fluctuation of the measurement-object gas to be introduced to the first internal cavity 20 becomes substantially negligible. The first internal cavity 20 is provided as a space for adjusting the oxygen partial pressure in the measurement-object gas introduced thereto via the second diffusion control section 13. The oxygen partial pressure is adjusted by actuating a main pump cell 21.

The main pump cell 21 is an electrochemical pump cell constituted of an inner pump electrode 22 including a ceiling electrode portion 22a disposed on the lower surface of the second solid electrolyte layer 6 over substantially the entirety of an area that faces the first internal cavity 20; an outer pump electrode 23 disposed on the upper surface of the second solid electrolyte layer 6 over an area that corresponds to the ceiling electrode portion 22a in such a manner as to be exposed to the outside of the element body 102; and the second solid electrolyte layer 6, the spacer layer 5, and the first solid electrolyte layer 4 that form a current path between the electrodes 22 and 23.

The inner pump electrode 22 is provided astride the upper and lower solid electrolyte layers (i.e., the second solid electrolyte layer 6 and the first solid electrolyte layer 4) that define the first internal cavity 20, and the spacer layer 5 that provides sidewalls of the first internal cavity 20. Specifically, the lower surface of the second solid electrolyte layer 6 that provides the ceiling surface of the first internal cavity 20 is provided with the ceiling electrode portion 22a, the upper surface of the first solid electrolyte layer 4 that provides the bottom surface of the first internal cavity 20 is provided with a bottom electrode portion 22b, and the ceiling electrode portion 22a and the bottom electrode portion 22b are connected to each other by lateral electrode portions (not illustrated) provided on respective sidewalls (inner surfaces) of the spacer layer 5 that form opposite sidewalls of the first internal cavity 20. Thus, the inner pump electrode 22 has a tunnel-like structure in a region where the lateral electrode portions are provided.

In the main pump cell 21, a desired voltage Vp0 is applied between the inner pump electrode 22 and the outer pump electrode 23, whereby a pump current Ip0 is caused to flow in the positive direction or the negative direction between the inner pump electrode 22 and the outer pump electrode 23. Thus, the oxygen in the first internal cavity 20 is pumped out to the external space, or the oxygen in the external space is pumped into the first internal cavity 20.

Furthermore, in order to detect the oxygen concentration (oxygen partial pressure) in the atmosphere within the first internal cavity 20, the inner pump electrode 22, the second solid electrolyte layer 6, the spacer layer 5, the first solid electrolyte layer 4, the third substrate layer 3, and the reference electrode 42 constitute an electrochemical sensor cell, that is, a main-pump-control oxygen-partial-pressure detection sensor cell 80.

The oxygen concentration (oxygen partial pressure) in the first internal cavity 20 is to be determined by measuring an electromotive force (voltage V0) in the main-pump-control oxygen-partial-pressure detection sensor cell 80. Furthermore, feedback control is performed on the voltage Vp0 of a variable power source 24 such that the voltage V0 becomes a target value, whereby the pump current Ip0 is controlled. Thus, the oxygen concentration in the first internal cavity 20 is maintained at a predetermined fixed value.

The third diffusion control section 30 is provided for applying a predetermined diffusion resistance to the measurement-object gas having an oxygen concentration (oxygen partial pressure) controlled in the first internal cavity 20 under the operation of the main pump cell 21, and then guiding the measurement-object gas to the second internal cavity 40.

The second internal cavity 40 is provided as a space for an auxiliary pump cell 50 to further adjust the oxygen partial pressure in the measurement-object gas having an oxygen concentration (oxygen partial pressure) preliminarily adjusted in the first internal cavity 20 and introduced to the second internal cavity 40 via the third diffusion control section 30. Thus, the oxygen concentration in the second internal cavity 40 is maintained at a fixed level with high accuracy, which enables the gas sensor 100 to achieve highly accurate NOx concentration measurement.

The auxiliary pump cell 50 is an auxiliary electrochemical pump cell constituted of an auxiliary pump electrode 51 including a ceiling electrode portion 51a disposed on the lower surface of the second solid electrolyte layer 6 over substantially the entirety of an area that faces the second internal cavity 40; the outer pump electrode 23 (but not limited to the outer pump electrode 23 and may possibly be an appropriate electrode disposed on the outer peripheral surface of the element body 102); and the second solid electrolyte layer 6, the spacer layer 5, and the first solid electrolyte layer 4.

The auxiliary pump electrode 51 is disposed in the second internal cavity 40 and has a tunnel-like structure similar to the inner pump electrode 22 disposed in the first internal cavity 20. Specifically, the second solid electrolyte layer 6 that provides the ceiling surface of the second internal cavity 40 is provided with the ceiling electrode portion 51a, the first solid electrolyte layer 4 that provides the bottom surface for the second internal cavity 40 is provided with a bottom electrode portion 51b, and the ceiling electrode portion 51a and the bottom electrode portion 51b are connected to each other by lateral electrode portions (not illustrated) provided on opposite wall surfaces of the spacer layer 5 that provide respective sidewalls of the second internal cavity 40. Thus, the auxiliary pump electrode 51 has a tunnel-like structure.

In the auxiliary pump cell 50, a desired voltage Vp1 is applied between the auxiliary pump electrode 51 and the outer pump electrode 23 so that the oxygen in the atmosphere within the second internal cavity 40 can be pumped out to the external space or the oxygen can be pumped into the second internal cavity 40 from the external space.

Furthermore, in order to control the oxygen partial pressure in the atmosphere within the second internal cavity 40, the auxiliary pump electrode 51, the reference electrode 42, the second solid electrolyte layer 6, the spacer layer 5, the first solid electrolyte layer 4, and the third substrate layer 3 constitute an electrochemical sensor cell, that is, an auxiliary-pump-control oxygen-partial-pressure detection sensor cell 81.

A variable power source 52 whose voltage is controlled based on an electromotive force (voltage V1) detected by the auxiliary-pump-control oxygen-partial-pressure detection sensor cell 81 causes the auxiliary pump cell 50 to perform pumping. Thus, the oxygen partial pressure in the atmosphere in the second internal cavity 40 is controlled to be a low partial pressure that has substantially no effect on NOx measurement.

Meanwhile, a pump current Ip1 generated by the auxiliary pump cell 50 is used in controlling the electromotive force of the main-pump-control oxygen-partial-pressure detection sensor cell 80. Specifically, the pump current Ip1 is input as a control signal to the main-pump-control oxygen-partial-pressure detection sensor cell 80, and the above target value for the voltage V0 of the main-pump-control oxygen-partial-pressure detection sensor cell 80 is controlled, whereby the gradient of the oxygen partial pressure in the measurement-object gas to be introduced to the second internal cavity 40 from the third diffusion control section 30 is controlled to be constantly fixed. To use the gas sensor 100 as a NOx sensor, the oxygen concentration in the second internal cavity 40 is maintained at a fixed value of about 0.001 ppm under the cooperation of the main pump cell 21 and the auxiliary pump cell 50.

The fourth diffusion control section 60 is provided for applying a predetermined diffusion resistance to the measurement-object gas having an oxygen concentration (oxygen partial pressure) controlled in the second internal cavity 40 under the operation of the auxiliary pump cell 50, and then guiding the measurement-object gas to the third internal cavity 61. The fourth diffusion control section 60 has a role of limiting the amount of NOx flowing into the third internal cavity 61.

The third internal cavity 61 is provided as a space where a process for measuring the concentration of nitrogen oxide (NOx) in the measurement-object gas is performed on the measurement-object gas having an oxygen concentration (oxygen partial pressure) preliminarily adjusted in the second internal cavity 40 and introduced to the third internal cavity 61 via the fourth diffusion control section 60. The NOx concentration is measured mainly in the third internal cavity 61 under the operation of a measurement pump cell 41.

The measurement pump cell 41 is configured to measure the NOx concentration in the measurement-object gas in the third internal cavity 61. The measurement pump cell 41 is an electrochemical pump cell constituted of the measurement electrode 44 disposed on the upper surface of the first solid electrolyte layer 4 in an area that faces the third internal cavity 61; and the outer pump electrode 23, the second solid electrolyte layer 6, the spacer layer 5, and the first solid electrolyte layer 4. The measurement electrode 44 also functions as a NOx reduction catalyst that reduces the NOx existing in the atmosphere in the third internal cavity 61.

In the measurement pump cell 41, oxygen resulting from the decomposition of the nitrogen oxide (NOx) in the atmosphere surrounding the measurement electrode 44 is pumped out, and the amount of oxygen thus produced is detected as a pump current Ip2.

Furthermore, in order to detect the oxygen partial pressure around the measurement electrode 44, the first solid electrolyte layer 4, the third substrate layer 3, the measurement electrode 44, and the reference electrode 42 constitute an electrochemical sensor cell, that is, a measurement-pump-control oxygen-partial-pressure detection sensor cell 82. A variable power source 46 is controlled based on an electromotive force (voltage V2) detected by the measurement-pump-control oxygen-partial-pressure detection sensor cell 82.

The measurement-object gas introduced to the second internal cavity 40 reaches the measurement electrode 44 in the third internal cavity 61 via the fourth diffusion control section 60 under a condition where the oxygen partial pressure is controlled. The nitrogen oxide (NOx) in the measurement-object gas surrounding the measurement electrode 44 is reduced (2NO→N₂+O₂) to produce oxygen. Then, the oxygen thus produced undergoes pumping by the measurement pump cell 41. During the pumping of the oxygen, a voltage Vp2 of the variable power source 46 is controlled such that the voltage V2 detected by the measurement-pump-control oxygen-partial-pressure detection sensor cell 82 becomes a fixed value (i.e., a target value). Because the amount of oxygen produced around the measurement electrode 44 is proportional to the concentration of the nitrogen oxide in the measurement-object gas, the nitrogen oxide concentration in the measurement-object gas is calculated by using the pump current Ip2 of the measurement pump cell 41.

Alternatively, if the measurement electrode 44, the first solid electrolyte layer 4, the third substrate layer 3, and the reference electrode 42 are combined into an electrochemical sensor cell serving as an oxygen-partial-pressure detection device, an electromotive force corresponding to the difference between the amount of oxygen resulting from the reduction of the NOx component in the atmosphere around the measurement electrode 44 and the amount of oxygen contained in the reference air is detectable. Thus, the concentration of the NOx component in the measurement-object gas may be obtained.

Furthermore, the second solid electrolyte layer 6, the spacer layer 5, the first solid electrolyte layer 4, the third substrate layer 3, the outer pump electrode 23, and the reference electrode 42 constitute an electrochemical sensor cell 83. Using an electromotive force (voltage Vref) generated by the sensor cell 83, the oxygen partial pressure in the measurement-object gas outside the element body 102 of the sensor element 101, specifically, around the outer pump electrode 23, is detectable.

In the gas sensor 100 having the above configuration, the measurement pump cell 41 receives the measurement-object gas whose oxygen partial pressure is constantly maintained at a fixed low value (i.e., a value that substantially has no effect on NOx measurement) as a result of actuation of the main pump cell 21 and the auxiliary pump cell 50. Thus, the NOx concentration in the measurement-object gas can be ascertained based on the pump current Ip2 flowing as a result of oxygen produced by NOx reduction being pumped out by the measurement pump cell 41 substantially in proportion to the NOx concentration in the measurement-object gas.

Here, the electrodes 22, 23, 42, 44, and 51 will be described. The inner pump electrode 22, the auxiliary pump electrode 51, and the measurement electrode 44 each contain Class 1 noble metal with catalytic activity. The Class 1 noble metal may be at least one of Pt, Rh, Ir, Ru, and Pd, for example. The outer pump electrode 23 and the reference electrode 42 each also contain Class 1 noble metal. The inner pump electrode 22 and the auxiliary pump electrode 51 each further contain Class 2 noble metal that reduces the catalytic activity of the Class 1 noble metal on the specific gas (NOX). Thus, the reducing ability of each of the inner pump electrode 22 and the auxiliary pump electrode 51 with respect to the NOx component in the measurement-object gas is weakened. The Class 2 noble metal may be Au, for example. The measurement electrode 44 does not contain any Class 2 noble metal. Thus, the reducing ability of the measurement electrode 44 with respect to the NOx component in the measurement-object gas is made higher than that of the inner pump electrode 22 and the auxiliary pump electrode 51. The measurement electrode 44 preferably contains at least one of Pt and Rh among Class 1 noble metals, or may contain both Pt and Rh. The outer pump electrode 23 and the reference electrode 42 each preferably contain none of Class 2 noble metals. The electrodes 22, 23, 42, 44, and 51 are each preferably a cermet that contains noble metal and oxygen-ion-conductive oxide (such as ZrO₂). Preferably, the electrodes 22, 23, 42, 44, and 51 are each a porous body. In the present embodiment, the inner pump electrode 22 and the auxiliary pump electrode 51 are each a porous cermet electrode composed of Pt and ZrO₂ with 1% of Au. The outer pump electrode 23 and the reference electrode 42 are each a porous cermet electrode composed of Pt and ZrO₂. The measurement electrode 44 is a porous cermet electrode composed of Pt, Rh, and ZrO₂.

In order to enhance oxygen ion conductivity of the solid electrolyte in the element body 102, the heater section 70 has a role of temperature adjustment for keeping the sensor element 101 warm by heating the sensor element 101. The heater section 70 includes a heater 71a, a heater insulation layer 71b, and a pressure release hole 71c.

The heater 71a is an electrical resistor interposed between the second substrate layer 2 and the third substrate layer 3 from above and below. The heater 71a generates heat when supplied with electricity from a heater power source 72, thereby heating and keeping warm the solid electrolyte in the element body 102 of the sensor element 101. Furthermore, the heater 71a is embedded in such a manner as to overlap the entire region from the first internal cavity 20 to the third internal cavity 61, and is capable of adjusting the entirety of the sensor element 101 to a temperature at which the solid electrolyte is activated.

The heater insulation layer 71b is provided on the upper and lower surfaces of the heater 71a and is an insulator composed of alumina or the like. The heater insulation layer 71b is provided for the purpose of obtaining electrical insulation between the second substrate layer 2 and the heater 71a and electrical insulation between the third substrate layer 3 and the heater 71a.

The pressure release hole 71c extends through the third substrate layer 3 and the reference-gas introduction layer 48 in such a manner as to communicate with the reference-gas introduction space 43. The pressure release hole 71c is provided for the purpose of alleviating an increase in internal pressure occurring with a temperature increase in the heater insulation layer 71b.

The gas sensor 100 further includes the control device 95. As illustrated in FIG. 3, the control device 95 includes the above-described variable power sources 24, 46, and 52, the above-described heater power source 72, and a controller 96. The controller 96 is a microprocessor including a CPU 97, a storage unit 98, and so forth. The storage unit 98 is an information-rewritable nonvolatile memory and is capable of storing, for example, various programs and various data. The controller 96 is configured to receive the voltage V0 of the main-pump-control oxygen-partial-pressure detection sensor cell 80, the voltage V1 of the auxiliary-pump-control oxygen-partial-pressure detection sensor cell 81, the voltage V2 of the measurement-pump-control oxygen-partial-pressure detection sensor cell 82, the voltage Vref of the sensor cell 83, the pump current Ip0 flowing through the main pump cell 21, the pump current Ip1 flowing through the auxiliary pump cell 50, and the pump current Ip2 flowing through the measurement pump cell 41. The controller 96 is configured to output control signals to the variable power sources 24, 46, and 52 and thus control the voltages Vp0, Vp1, and Vp2 that are to be output by the variable power sources 24, 46, and 52, thereby controlling the main pump cell 21, the measurement pump cell 41, and the auxiliary pump cell 50. The controller 96 is configured to output a control signal to the heater power source 72, thereby controlling the electric power to be supplied from the heater power source 72 to the heater 71a. The storage unit 98 further stores target values V0*, V1*, V2*, and the like, to be described below. The CPU 97 of the controller 96 is configured to refer to the target values V0*, V1*, and V2* and thus control the pump cells 21, 41, and 50.

The controller 96 is configured to perform an auxiliary-pump control process in which the auxiliary pump cell 50 is controlled such that the oxygen concentration in the second internal cavity 40 becomes a target concentration. Specifically, the controller 96 performs feedback control on the voltage Vp1 of the variable power source 52 in such a manner as to set the voltage V1 to a fixed value (referred to as “target value V1*”), thereby controlling the auxiliary pump cell 50. The target value V1* is defined as a value that makes the oxygen concentration in the second internal cavity 40 a predetermined low concentration that has substantially no effect on NOx measurement.

The controller 96 is configured to perform a main-pump control process in which the main pump cell 21 is controlled such that the pump current Ip1 generated when the auxiliary pump cell 50 adjusts the oxygen concentration in the second internal cavity 40 in the auxiliary-pump control process becomes a target current (referred to as “target value Ip1*”). Specifically, the controller 96 sets (i.e., performs feedback control on), based on the pump current Ip1, the target value for the voltage V0 (referred to as “target value V0*”) in such a manner as to set the pump current Ip1 flowing at the voltage Vp1 to a fixed target value Ip1*. Furthermore, the controller 96 performs feedback control on the voltage Vp0 of the variable power source 24 in such a manner as to set the voltage V0 to the target value V0* (i.e., to set the oxygen concentration in the first internal cavity 20 to a target concentration). Through the main-pump control process, the gradient of the oxygen partial pressure in the measurement-object gas to be introduced from the third diffusion control section 30 to the second internal cavity 40 is constantly fixed. The target value V0* is set to a value at which the oxygen concentration in the first internal cavity 20 is higher than 0% and is a low concentration. The pump current Ip0 that flows during the main-pump control process changes with the oxygen concentration in the measurement-object gas that flows from the gas inlet 10 into the measurement-object gas flow section (i.e., the measurement-object gas around the sensor element 101). Therefore, the controller 96 is also capable of detecting the oxygen concentration in the measurement-object gas based on the pump current Ip0.

The above main-pump control process and auxiliary-pump control process are also collectively referred to as “adjustment-pump control process”. Furthermore, the first internal cavity 20 and the second internal cavity 40 are also collectively referred to as “oxygen-concentration adjustment chamber”. The main pump cell 21 and the auxiliary pump cell 50 are also collectively referred to as “adjustment pump cell”. In the adjustment-pump control process performed by the controller 96, the adjustment pump cell adjusts the oxygen concentration in the oxygen-concentration adjustment chamber.

The controller 96 is configured to perform a measurement-pump control process in which the measurement pump cell 41 is controlled such that the voltage V2 becomes a fixed value (referred to as “target value V2*”) (i.e., such that the oxygen concentration in the third internal cavity 61 becomes a predetermined low concentration). Specifically, the controller 96 performs feedback control on the voltage Vp2 of the variable power source 46 in such a manner as to set the voltage V2 to the target value V2*, thereby controlling the measurement pump cell 41. Through the measurement-pump control process, oxygen is pumped out from the third internal cavity 61.

Through the measurement-pump control process, oxygen produced as a result of the NOx in the measurement-object gas being reduced in the third internal cavity 61 is pumped out from the third internal cavity 61 such that the oxygen becomes substantially zero. Then, the controller 96 acquires the pump current Ip2 as a value detected in correspondence with the oxygen produced in the third internal cavity 61 from the specific gas (in this case, NOx), and calculates the NOx concentration in the measurement-object gas based on the pump current Ip2.

The storage unit 98 stores, for example, a relational expression (e.g., a linear function or quadratic function expression) or a map that represents the correspondence relationship between the pump current Ip2 and the NOx concentration. Such a relational expression or map can be preliminarily obtained through experiments.

The controller 96 is configured to perform a heater control process in which a control signal is output to the heater power source 72 so that the temperature of the heater 71a is controlled to be a target temperature (800° C., for example). Here, the temperature of the heater 71a can be expressed as a linear function of the resistance value of the heater 71a. In the heater control process, the controller 96 calculates the resistance value of the heater 71a as a value equivalent to the temperature of the heater 71a (as a value convertible into temperature), and performs feedback control on the heater power source 72 such that the resistance value thus calculated becomes a target resistance value (a resistance value corresponding to the target temperature). The controller 96 is capable of acquiring, for example, the voltage of the heater 71a and the current flowing through the heater 71a and calculating the resistance value of the heater 71a based on the voltage and current thus acquired. Alternatively, the controller 96 may calculate the resistance value of the heater 71a through, for example, a three-terminal method or a four-terminal method. In powering the heater 71a, the heater power source 72 changes, based on the control signal from the controller 96 for example, the value of the voltage to be applied to the heater 71a, thereby adjusting the electric power to be supplied to the heater 71a.

The control device 95 inclusive of the variable power sources 24, 46, and 52, the heater power source 72, and so forth illustrated in FIG. 3 is connected to the electrodes 22, 23, 42, 44, and 51 and the heater 71a via respective conducting sections 74. The plurality of conducting sections 74 each include a connector electrode 75 and a lead 76. The lead 76 allows the connector electrode 75 and a corresponding one of the electrodes to be electrically continuous with each other. In FIG. 4, all of the plurality of connector electrodes 75 are illustrated, whereas only one of the leads 76 that is provided for the measurement electrode 44 is illustrated.

The plurality of connector electrodes 75 each function as a terminal that allows the sensor element 101 and an external element to be electrically continuous with each other. As illustrated in FIG. 4, the plurality of connector electrodes 75 are each disposed at a rear end part of the first face 102a (upper face) or the second face 102b (lower face) of the element body 102 of the sensor element 101. Specifically, the plurality of connector electrodes 75 are the following: connector electrodes 75a to 75d disposed at the rear end part of the first face 102a of the element body 102 in that order from the left side, and connector electrodes 75e to 75h disposed at the rear end part of the second face 102b of the element body 102 in that order from the left side. The connector electrodes 75a to 75d and 75h are connected to (electrically continuous with) the measurement electrode 44, the outer pump electrode 23, the auxiliary pump electrode 51, the inner pump electrode 22, and the reference electrode 42 via the respective leads 76. The connector electrodes 75e to 75g are connected to the heater 71a via the respective leads 76.

Here, details of the measurement electrode 44 and the corresponding conducting section 74 (the connector electrode 75a and the lead 76) will be described with reference to FIGS. 4 and 5. Between each two of the layers 1 to 6 of the element body 102 that are adjacent in the layered direction (up-down direction) is provided a hermetic bonding layer, not illustrated in FIG. 2. Accordingly, each adjacent two layers are bonded to each other with the corresponding bonding layer. The bonding layers preferably have oxygen ion conductivity, as with the layers 1 to 6. In the present embodiment, the bonding layers are each made of ceramic chiefly composed of zirconia, as with the layers 1 to 6. As illustrated in FIG. 5, a bonding layer 7 among the bonding layers bonds the first solid electrolyte layer 4 and the spacer layer 5 to each other. The bonding layer 7 covers substantially the entirety of the upper surface of the first solid electrolyte layer 4, excluding the measurement-object gas flow section constituted of the buffer space 12, the first internal cavity 20, the second internal cavity 40, and so forth.

The measurement electrode 44 is disposed on the upper surface of the first solid electrolyte layer 4 in an area that faces the third internal cavity 61. The lead 76 provided for the measurement electrode 44 includes an inner lead 77 and a peripheral-face lead 78. The inner lead 77 is disposed inside the element body 102 and is connected to (electrically continuous with) the measurement electrode 44 and the peripheral-face lead 78. Specifically, the inner lead 77 includes first to fourth segments 77a to 77d. The first segment 77a is disposed on the upper surface of the first solid electrolyte layer 4 in an area that faces the third internal cavity 61. The right end of the first segment 77a is connected to the measurement electrode 44. The first segment 77a extends linearly in the left-right direction up to the left end of the third internal cavity 61. The right end of the second segment 77b is connected to the left end of the first segment 77a. The second segment 77b extends linearly in the left-right direction but does not reach the left end of the element body 102. The front end of the third segment 77c is connected to the left end of the second segment 77b. The third segment 77c extends linearly in the front-rear direction up to a position near the rear end of the element body 102. The right end of the fourth segment 77d is connected to the rear end of the third segment 77c. The fourth segment 77d extends linearly in the left-right direction up to the left end of the element body 102. The left end of the fourth segment 77d reaches (is routed to) the third face 102c (left end face) of the element body 102 and is covered by and connected to the peripheral-face lead 78. The inner lead 77 is a conductor chiefly composed of a noble metal such as platinum (Pt) or a high-melting-point metal such as tungsten (W) or molybdenum (Mo). The inner lead 77 is preferably a cermet conductor containing a noble metal or a high-melting-point metal and zirconia that is the same as the chief component of the first solid electrolyte layer 4.

In the inner lead 77, the entirety of the second and third segments 77b and 77c and the outer periphery of a part of the fourth segment 77d excluding a left end part thereof are enclosed by a lead insulation layer 79. The lead insulation layer 79 insulates the portion of the inner lead 77 whose outer circumference is surrounded by the lead insulation layer 79 from the first solid electrolyte layer 4 and the spacer layer 5. Note that the outer periphery of the left end part of the fourth segment 77d is not enclosed by the lead insulation layer 79. That is, during the manufacture of the sensor element 101, the outer periphery of the left end part of the fourth segment 77d is prevented from being enclosed by the lead insulation layer 79. Therefore, the connection (electrical continuity) between the left end of the fourth segment 77d and the peripheral-face lead 78 is less likely to be hindered. The lead insulation layer 79 is an insulator composed of ceramic such as alumina.

The peripheral-face lead 78 is disposed near the rear end of the third face 102c of the element body 102 and is connected to the inner lead 77 and the connector electrode 75a. Specifically, the peripheral-face lead 78 is disposed on the third face 102c in such a manner as to cover the entirety of the left end of the fourth segment 77d of the inner lead 77, thereby preventing the left end of the fourth segment 77d from being exposed to the outside of the sensor element 101. The peripheral-face lead 78 is in contact with the solid electrolyte layers. The peripheral-face lead 78 is disposed astride the solid electrolyte layers and corresponding ones of the bonding layers of the element body 102. A central part of the right end face of the peripheral-face lead 78 is connected to (electrically continuous with) the left end of the fourth segment 77d. The upper end of the right end face of the peripheral-face lead 78 is connected to a front end part of the left end face of the connector electrode 75a.

The peripheral-face lead 78 is a conductor having a dense composition with noble metal, alumina (Al₂O₃), and silica (SiO₂). A specific example of noble metal is platinum (Pt) or a platinum alloy. Since the peripheral-face lead 78 contains not only noble metal but also alumina and silica, the peripheral-face lead 78 has increased adhesiveness. Furthermore, since the peripheral-face lead 78 contains silica, the peripheral-face lead 78 has increased denseness. Thus, both denseness and adhesiveness of the peripheral-face lead 78 are increased. The present inventors have demonstrated such facts through experiments, analyses, and the like. The “dense composition” refers to a composition in which, letting the porosity of the peripheral-face lead 78 be Rp [%], Rp is lower than 3.0%. The peripheral-face lead 78 preferably has a porosity Rp of lower than 0.5%.

If some kind of gas outside the sensor element 101 (element body 102) enters the inside of the element body 102 through the fourth segment 77d of the inner lead 77 or through a gap between the fourth segment 77d and the element body 102, the gas may move toward the measurement electrode 44 along the inner lead 77 or the gap between the inner lead 77 and the element body 102 (a gap between the inner lead 77 and the lead insulation layer 79 or a gap between the lead insulation layer 79 and the element body 102) and reach the measurement electrode 44. Examples of some kind of gas outside the sensor element 101 include a volatile organic gas generated from the rubber stopper 157 if, for example, the sensor element 101 is exposed to a high-temperature environment; a slight amount of the measurement-object gas that has entered the space 149 from the sensor element chamber 133; and any gas, air (for example, the reference gas in the space 149), or vapor that has entered the inside of the external cylinder 148 from outside the gas sensor 100 through a gap between the rubber stopper 157 and the external cylinder 148. If such a gas reaches the measurement electrode 44, the oxygen concentration around the measurement electrode 44 changes. Such a change may change the voltage V2 to be detected by the measurement-pump-control oxygen-partial-pressure detection sensor cell 82, the amount of oxygen to be pumped out from the third internal cavity 61 in the measurement-pump control process, and the pump current Ip2. For example, if a volatile organic gas generated from the rubber stopper 157 reaches the measurement electrode 44, the oxygen concentration around the measurement electrode 44 may be reduced, leading to an increase in the voltage V2 and a decrease in the pump current Ip2. That is, if some kind of gas reaches the measurement electrode 44 and changes the pump current Ip2, the accuracy in the detection of concentration of the specific gas (NOx) may be deteriorated. In view of such circumstances, the present embodiment provides increased denseness to a part that covers the inner lead 77, i.e., the peripheral-face lead 78 forming a part of the conducting section 74 that contributes to the less likeliness of the gas reaching the measurement electrode 44. Thus, some kind of gas outside the element body 102 is made less likely to pass through the peripheral-face lead 78, to enter the inside of the element body 102 through the fourth segment 77d of the inner lead 77 or a gap between the fourth segment 77d and the element body 102, and to reach the measurement electrode 44. Consequently, such a gas is made less likely to change the pump current Ip2. Therefore, the accuracy in the detection of concentration of the specific gas (NOx) is made less likely to be deteriorated. Furthermore, the less likeliness of such a gas reaching the measurement electrode 44 makes the measurement electrode 44 less likely to be deteriorated.

Furthermore, since the peripheral-face lead 78 has increased adhesiveness, when the sensor element 101 contracts by being calcinated in the manufacture thereof, the peripheral-face lead 78 is less likely to peel from the third face 102c of the element body 102. Furthermore, thermal expansion or contraction of the sensor element 101 is less likely to peel the peripheral-face lead 78 from the third face 102c of the element body 102.

Letting the volumes of noble metal, alumina, and silica contained in the peripheral-face lead 78 be Vp [vol %], Va [vol %], and Vs [vol %], respectively, the peripheral-face lead 78 may be as follows. The peripheral-face lead 78 preferably satisfies Va/Vs≤1.5. Thus, the denseness of the peripheral-face lead 78 is further increased. The peripheral-face lead 78 may satisfy 0.1≤Va/Vs. The peripheral-face lead 78 preferably satisfies 1.5≤Vp/(Va+Vs). Thus, a more assured effect of increasing the denseness of the peripheral-face lead 78 is obtained. In such a case, it is more preferable to satisfy 2.6≤Vp/(Va+Vs). Thus, the denseness of the peripheral-face lead 78 is further increased. The peripheral-face lead 78 may satisfy Vp/(Va+Vs)≤19.0. It is more preferable that the peripheral-face lead 78 satisfy Va/Vs≤1.5 and 1.5≤Vp/(Va+Vs). It is much more preferable that the peripheral-face lead 78 satisfy Va/Vs≤1.5 and 2.6≤Vp/(Va+Vs). The volume Vp may be 60 vol % or greater. The volume Vp may be 95 vol % or smaller. The volume Va may be 1 vol % or greater. The volume Va may be 15 vol % or smaller. The volume Vs may be 2 vol % or greater or may be 4 vol % or greater. The volume Vs may be 25 vol % or smaller.

The ratio of the total mass of alkali metal and alkaline-earth metal contained in the peripheral-face lead 78 on an oxide basis preferably satisfies 0.1 wt % or smaller. Here, it is also conceivable to increase the denseness and the adhesiveness by employing a peripheral-face lead 78 containing noble metal and a glass component, instead of the peripheral-face lead 78 containing noble metal, alumina, and silica. In such a case, however, the peripheral-face lead 78 contains as a glass component a large amount of alkali-metal oxide and/or alkaline-earth-metal oxide, such as sodium oxide (Na₂O), calcium oxide (Cao), or magnesium oxide (MgO). In such a case, for example, the voltage Vp2 to be applied when the sensor element 101 is used causes the alkali metal and/or alkaline-earth metal to move by undergoing electromigration (for example, to move from the inside of the peripheral-face lead 78 to the inner lead 77), which lowers the conductivity (increases the resistance value) of the peripheral-face lead 78. Accordingly, the pump current Ip2 is lowered, leading to an accuracy deterioration in the detection of concentration of the specific gas (NOx). In view of such circumstances, the peripheral-face lead 78 according to the present embodiment contains alumina and silica to have increased adhesiveness and increased denseness. Furthermore, since the ratio of the total mass of alkali metal and alkaline-earth metal on an oxide basis is 0.1 wt % or smaller, the probability of accuracy deterioration in the detection of concentration of the specific gas that may be caused by such electromigration is lowered.

Letting the thickness of the peripheral-face lead 78 be Dc [mm], the porosity Rp and the thickness Dc of the peripheral-face lead 78 may satisfy Rp/Dc≤145%/mm. The peripheral-face lead 78 may satisfy Rp/Dc≤120%/mm. The peripheral-face lead 78 may satisfy Rp/Dc≥1.5%/mm. The peripheral-face lead 78 may satisfy Rp/Dc≥4.5%/mm. The porosity Rp of the peripheral-face lead 78 may be 0.01% or higher. The thickness Dc of the peripheral-face lead 78 may be 0.001 mm or greater. The thickness Dc of the peripheral-face lead 78 may be 0.080 mm or smaller.

The porosity, such as the porosity Rp of the peripheral-face lead 78, is defined as a value derived as follows by using an image (SEM image) obtained in an observation through a scanning electron microscope (SEM). First, a measurement object is cut to have a section (if the measurement object is the peripheral-face lead 78, a section taken in the thickness direction of the peripheral-face lead 78). The section, regarded as an observation surface, is embedded with resin and is polished, whereby an observation sample is obtained. Subsequently, the observation surface of the observation sample is photographed by SEM photography (with a secondary electron image, an acceleration voltage of 15 kV, and a magnifying power of 1000, but a magnifying power higher than 1000 and lower than or equal to 5000 if the magnifying power of 1000 is inappropriate), whereby a SEM image of the measurement object is obtained. Subsequently, the obtained image is analyzed, so that a threshold value is determined using the discriminant analysis method (Otsu binarization method) from a brightness distribution of brightness data on the pixels in the image. Then, each pixel in the image is binarized into an object section and a pore section based on the determined threshold value, and the area of the object section and the area of the pore section are calculated. Furthermore, the percentage of the area of the pore section relative to the overall area (i.e., the total area of the object section and the pore section) is derived as the porosity (in %).

The volumes Vp [vol %], Va [vol %], and Vs [vol %] of noble metal, alumina, and silica contained in the peripheral-face lead 78 are defined as a value derived through SEM-EDX (EDX: energy-dispersive X-ray spectroscopy) as follows. First, as with the above case of measurement of the porosity Rp, a measurement object is cut to have a section regarded as an observation surface, whereby an observation sample is obtained. Subsequently, element mapping through SEM-EDX is performed on a plurality of locations (three locations, for example) of the observation surface of the observation sample. The result of the element mapping is used to calculate the average element concentration for each of noble metal, aluminum, and silicon. Based on the calculated average element concentration of noble metal, the volume Vp [vol %] is calculated. Furthermore, the average element concentration of aluminum is subjected to oxide conversion to calculate the volume Va [vol %], and the average element concentration of silicon is subjected to oxide conversion to calculate the volume Vs [vol %].

The ratio of the total mass of alkali metal and alkaline-earth metal contained in the peripheral-face lead 78 on an oxide basis is defined as a value derived through ICP-AES (inductively coupled plasma atomic emission spectroscopy) as follows. First, a measurement object is peeled from the sensor element 101 and is weighed to a predetermined amount for use as a measurement sample. Subsequently, the measurement sample is melted with sulfuric acid into a solution. The solution is subjected to ICP-AES for qualitative analysis of elements contained in the measurement sample. Using a test Na concentration obtained in the qualitative analysis and a Na calibration curve obtained in advance, the actual Na concentration is calculated. The actual Na concentration thus obtained is subjected to oxide conversion to calculate the ratio of the mass of sodium oxide contained in the measurement object. The ratio of the mass of alkali metal and alkaline-earth metal other than Na on an oxide basis is also calculated in a similar manner. Using the results of these calculations, the ratio of the total mass of alkali metal and alkaline-earth metal contained in the measurement object on an oxide basis is calculated.

Now, an exemplary method of manufacturing the sensor element 101 included in the gas sensor 100 will be described. First, six non-calcinated ceramic green sheets each containing an oxygen-ion-conductive solid electrolyte, such as zirconia, as a ceramic component are prepared. In each of these green sheets, a plurality of sheet holes to be used for positioning during printing or stacking as well as necessary through-holes and the like are provided in advance. Furthermore, the green sheet that is to become the spacer layer 5 is preliminarily subjected to a punching process or the like in which a space that is to become the measurement-object gas flow section is provided. Likewise, the green sheet that is to become the first solid electrolyte layer 4 is subjected to a process for providing a space that is to become the reference-gas introduction space 43. Then, a pattern-printing process and a drying process for forming various patterns in the ceramic green sheets are performed in correspondence with the first substrate layer 1, the second substrate layer 2, the third substrate layer 3, the first solid electrolyte layer 4, the spacer layer 5, and the second solid electrolyte layer 6. Specific patterns to be formed include, for example, patterns that serve as electrodes such as the measurement electrode 44 described above, inner leads such as the inner leads 77 connected to the electrodes, lead insulation layers such as the lead insulation layers 79, the connector electrodes 75, the reference-gas introduction layer 48, and the heater section 70. The pattern-printing process is performed in which a pattern-forming paste prepared in accordance with the properties required for an object to be formed is applied onto a green sheet by using a known screen printing technique. The drying process is performed by using a known drying technique. When the pattern-printing process and the drying process are completed, a printing process and a drying process are performed in which a bonding paste that is to become the bonding layers (including the above bonding layer 7) for stacking and bonding together the green sheets corresponding to the respective layers are printed and dried. Subsequently, the green sheets provided with the bonding paste are stacked in a predetermined order while being positioned at the sheet holes, and are then subjected to a pressure bonding process in which the green sheets are put under predetermined temperature and pressure conditions to be pressure bonded into a single layered body. The layered body thus obtained contains a plurality of sensor elements 101 therein. The layered body is cut into pieces each having the size of the sensor element 101. Subsequently, a pattern that is to become the peripheral-face lead 78 is formed by screen printing on a section obtained by cutting the layered body, i.e., on a part that serves as the third face 102c of the element body 102 of the sensor element 101. Furthermore, the pattern is dried. Then, the layered body is calcinated at a predetermined calcination temperature, whereby a sensor element 101 is obtained.

To form patterns that are to become the inner lead 77 and the lead insulation layer 79 on the green sheet that is to become the first solid electrolyte layer 4, the following steps may alternatively be taken, for example. First, a pattern that is to become a part of the lead insulation layer 79 that covers the lower side of the inner lead 77 is formed on the green sheet. Subsequently, a pattern that is to become the inner lead 77 is formed. Then, parts of the lead insulation layer 79 that cover the lateral sides and upper side of the inner lead 77 are formed.

The pattern-forming paste for forming the peripheral-face lead 78 is a paste containing noble metal such as platinum, and alumina, silica, a binder, and a solvent. The pattern-forming paste may further be mixed with at least one of a plasticizer and a dispersion aid. The pattern-forming paste may be mixed with any other materials, instead of alumina and silica, that turn into alumina and silica by being calcinated. For example, one or more of aluminum hydroxide, silicic acid, mullite, and kaolinite may be mixed into the pattern-forming paste. The porosity Rp of the peripheral-face lead 78 is adjustable by, for example, adjusting the ratio of noble metal, alumina, and silica contained in the pattern-forming paste for forming the peripheral-face lead 78 and thus adjusting the above Va/Vs and/or Vp/(Va+Vs) of the peripheral-face lead 78 having undergone calcination. The thickness Dc of the peripheral-face lead 78 is adjustable by, for example, adjusting the viscosity of the pattern-forming paste for forming the peripheral-face lead 78 or changing the number of times of printing to be performed in the pattern-forming process.

Particles contained in the pattern-forming paste for forming the peripheral-face lead 78, particularly, particles of alumina and silica, may contain alkali metal and/or alkaline-earth metal as impurities. If such particles have high purity or if such particles are cleansed before being mixed with other ingredients for preparation of the pattern-forming paste, the ratio of the total mass of alkali metal and alkaline-earth metal contained in the peripheral-face lead 78 on an oxide basis may be set to 0.1 wt % or smaller.

Subsequently, a gas sensor 100 having the sensor element 101 incorporated therein is manufactured. For example, an element sealing unit 141 is attached to the sensor element 101 to seal and secure the sensor element 101, and a protection cover 130 is attached to a part of the element sealing unit 141 that is near the front end of the element body 102 of the sensor element 101. Furthermore, a connector 150 and lead wires 155 are attached to the rear end of the element body 102 of the sensor element 101 in such a manner as to be electrically continuous with the connector electrodes 75. Furthermore, an external cylinder 148 is attached to a part of the element sealing unit 141 that is near the rear end of the element body 102, the lead wires 155 are routed from the external cylinder 148 to the outside, and the external cylinder 148 is welded to the main metal fitting 142. Subsequently, the lead wires 155 are passed through the through-holes provided in the rubber stopper 157, the rubber stopper 157 is fitted into the external cylinder 148, and the external cylinder 148 is swaged such that the diameter thereof is reduced to form swaged sections 148a and 148b. Thus, the rubber stopper 157 and the external cylinder 148 are secured to each other. Then, a control device 95 and the sensor element 101 are connected to each other via the lead wires 155. Thus, a gas sensor 100 is obtained.

Now, an exemplary usage of the gas sensor 100 will be described. The CPU 97 in the controller 96 first controls the heater power source 72 to supply electric power to the heater 71a such that the temperature of the heater 71a becomes a target temperature (800° C., for example). The CPU 97 controls the temperature of the heater 71a by, for example, acquiring a value (for example, the resistance value or current value of the heater 71a) that is convertible into the temperature of the heater 71a and performing feedback control on the heater power source 72 based on the thus acquired value. When the temperature of the heater 71a reaches the target temperature (or near the target temperature), the CPU 97 starts controlling the above pump cells 21, 41, and 50 (the adjustment-pump control process and the measurement-pump control process) and acquiring the voltages V0, V1, V2, and Vref from the above sensor cells 80 to 83. When a measurement-object gas is introduced through the gas inlet 10 in this state, the measurement-object gas travels through the first diffusion control section 11, the buffer space 12, and the second diffusion control section 13 and reaches the first internal cavity 20. Then, the oxygen concentration in the measurement-object gas is adjusted in the first internal cavity 20 and the second internal cavity 40 by the main pump cell 21 and the auxiliary pump cell 50, and the measurement-object gas thus adjusted reaches the third internal cavity 61. Subsequently, the CPU 97 detects the NOx concentration in the measurement-object gas based on the acquired pump current Ip2 and the correspondence relationship stored in the storage unit 98.

Here, the correspondence between the elements according to the present embodiment and the elements according to the present invention is clarified. The first substrate layer 1, the second substrate layer 2, the third substrate layer 3, the first solid electrolyte layer 4, the spacer layer 5, and the second solid electrolyte layer 6 according to the present embodiment each correspond to the solid electrolyte layer according to the present invention. The element body 102 corresponds to the element body. The first to fourth faces 102a to 102d each correspond to the peripheral face. The measurement electrode 44 corresponds to the inner electrode. The conducting section 74 corresponds to the conducting section. The inner lead 77 corresponds to the inner conducting section. The connector electrode 75a and the peripheral-face lead 78 each correspond to the outer conducting section. The peripheral-face lead 78 corresponds to the dense segment. A combination of the protection cover 130 and the sensor assembly 140 corresponds to the case. The rubber stopper 157 corresponds to the sealing member.

In the sensor element 101 included in the gas sensor 100 according to the present embodiment described in detail above, the peripheral-face lead 78 covers the left end of the fourth segment 77d of the inner lead 77 and has a dense composition with noble metal, alumina, and silica. Therefore, both denseness and adhesiveness are increased in the peripheral-face lead 78, which is a part of the conducting section 74 that contributes to the less likeliness of the gas reaching the measurement electrode 44. Since the peripheral-face lead 78 is dense, some kind of gas, such as volatile organic gas generated from the rubber stopper 157, outside the sensor element 101 (element body 102) is less likely to pass through the peripheral-face lead 78, to enter the inside of the element body 102 through the fourth segment 77d of the inner lead 77 or a gap between the fourth segment 77d and the element body 102, and to reach the measurement electrode 44. Consequently, such a gas is less likely to change the pump current Ip2. Therefore, the accuracy in the detection of concentration of the specific gas (NOx) is less likely to be deteriorated. Furthermore, since the peripheral-face lead 78 has increased adhesiveness, when the sensor element 101 contracts by being calcinated in the manufacture of the sensor element 101, the peripheral-face lead 78 is less likely to peel from the third face 102c of the element body 102. Furthermore, thermal expansion or contraction of the sensor element 101 is less likely to peel the peripheral-face lead 78 from the third face 102c of the element body 102. Furthermore, even if the sensor element 101 having undergone calcination receives an external stress (contact with a jig or any other like incident) while being moved in the manufacturing process, the peripheral-face lead 78 is less likely to peel from the third face 102c.

In the sensor element 101, the peripheral-face lead 78 satisfies Va/Vs≤1.5. Thus, the denseness of the peripheral-face lead 78 is further increased, and some kind of gas outside the element body 102 is much less likely to pass through the peripheral-face lead 78.

In the sensor element 101, the peripheral-face lead 78 satisfies 1.5≤Vp/(Va+Vs). Thus, a more assured effect of increasing the denseness of the peripheral-face lead 78 is obtained. Furthermore, the peripheral-face lead 78 satisfies 2.6≤Vp/(Va+Vs). Thus, the denseness of the peripheral-face lead 78 is further increased.

In the sensor element 101, the ratio of the total mass of alkali metal and alkaline-earth metal contained in the peripheral-face lead 78 on an oxide basis satisfies 0.1 wt % or smaller. Such a composition lowers the probability of reduction in the conductivity of the peripheral-face lead 78 that may be caused by the electromigration of the alkali metal and/or alkaline-earth metal in the peripheral-face lead 78, and thus lowers the probability of accuracy deterioration in the detection of concentration of the specific gas that may be caused by the reduction in the conductivity.

The present invention is not limited whatsoever to the above embodiment, and various embodiments are conceivable so long as they belong to the technical scope of the present invention.

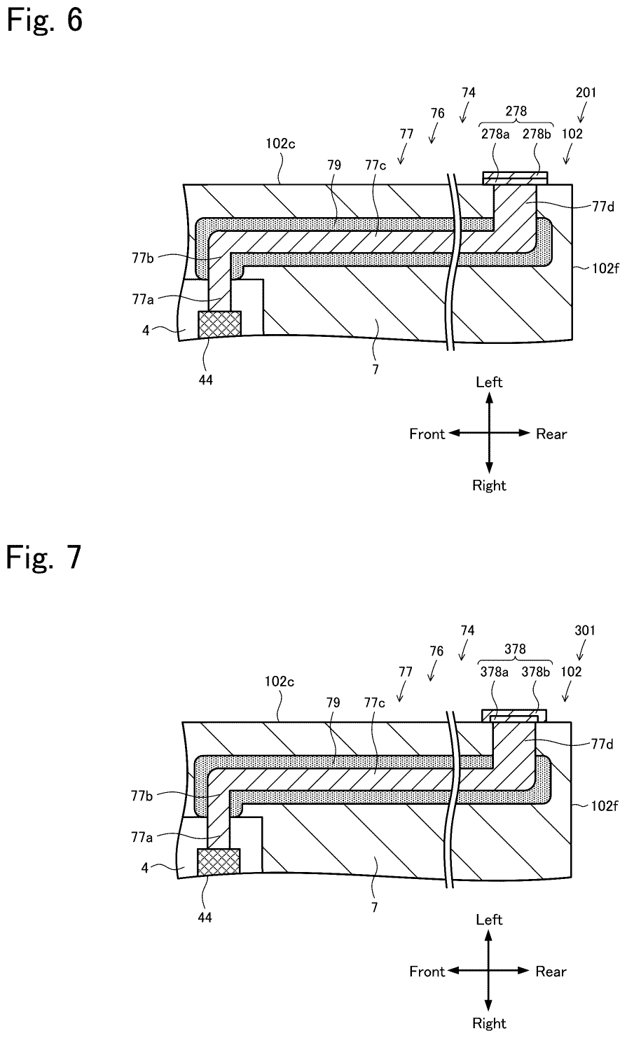

For example, while the peripheral-face lead 78 according to the above embodiment is constituted of a single layer, such an embodiment is not limiting. For example, as in a sensor element 201 according to a modification illustrated in FIG. 6 or a sensor element 301 according to another modification illustrated in FIG. 7, the peripheral-face lead 78 may be replaced with a peripheral-face lead 278 or 378. The peripheral-face leads 278 and 378 are each constituted of two layers. The peripheral-face leads 278 and 378 may each be constituted of three or more layers, instead of being constituted of two layers.

In the sensor element 201 illustrated in FIG. 6, the peripheral-face lead 278 generally has a substantially rectangular parallelepiped shape and includes a first peripheral-face lead 278a and a second peripheral-face lead 278b. The first peripheral-face lead 278a has a substantially rectangular parallelepiped shape and is disposed on the third face 102c (left face) of the element body 102 in such a manner as to cover the entirety of the left end of the fourth segment 77d of the inner lead 77, thereby preventing the left end of the fourth segment 77d from being exposed to the outside of the sensor element 101. The second peripheral-face lead 278b has a substantially rectangular parallelepiped shape and is disposed on the left end face of the first peripheral-face lead 278a. In such a case, the first peripheral-face lead 278a is the part of the conducting section 74 that contributes to the less likeliness of the gas reaching the measurement electrode 44. Therefore, the first peripheral-face lead 278a may be provided as the dense segment according to the present invention, as with the peripheral-face lead 78 of the sensor element 101.

In the sensor element 301 illustrated in FIG. 7, the peripheral-face lead 378 generally has a substantially rectangular parallelepiped shape and includes a first peripheral-face lead 378a and a second peripheral-face lead 378b. The first peripheral-face lead 378a has a substantially rectangular parallelepiped shape and is disposed on the third face 102c (left face) of the element body 102 in such a manner as to cover the entirety of the left end of the fourth segment 77d of the inner lead 77, thereby preventing the left end of the fourth segment 77d from being exposed to the outside of the sensor element 301. The second peripheral-face lead 378b covers five faces, excluding the right end face, of the first peripheral-face lead 378a and is in contact with the third face 102c of the element body 102, thereby preventing the fourth segment 77d of the inner lead 77 and the first peripheral-face lead 378a from being exposed to the outside of the sensor element 301. In such a case, the first peripheral-face lead 378a and the second peripheral-face lead 378b are each the part of the conducting section 74 that contributes to the less likeliness of the gas reaching the measurement electrode 44. Therefore, the first peripheral-face lead 378a may be provided as the dense segment according to the present invention, or the second peripheral-face lead 378b may be provided as the dense segment according to the present invention. Alternatively, the entirety of the peripheral-face lead 378 may be provided as the dense segment according to the present invention. Hence, any of the first peripheral-face lead 378a, the second peripheral-face lead 378b, and the entirety of the peripheral-face lead 378 that is provided as the dense segment according to the present invention may be configured the same as the peripheral-face lead 78 of the sensor element 101.

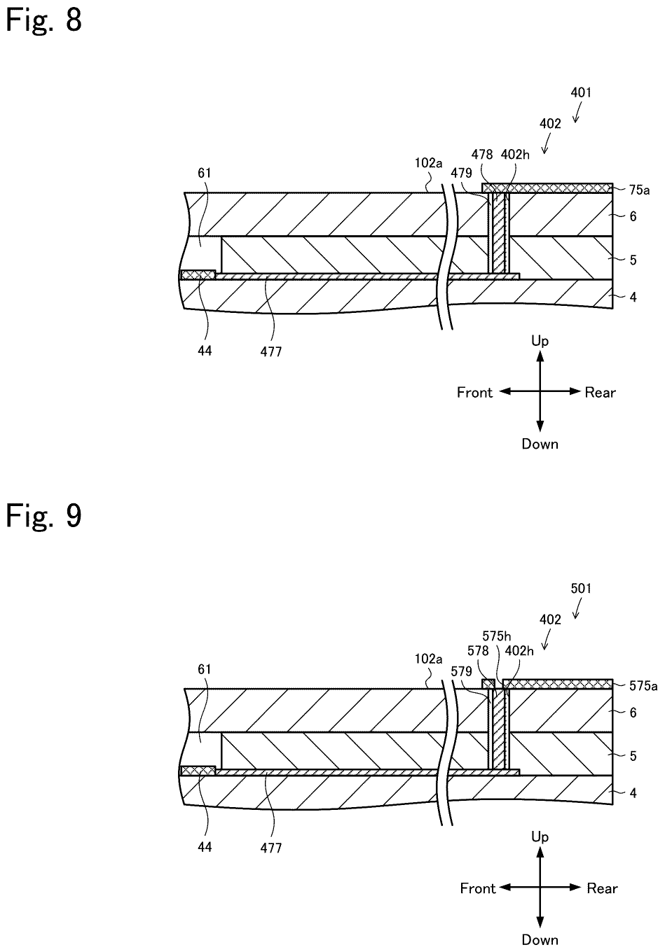

While the above embodiment relates to a case where the connector electrode 75a is connected to the measurement electrode 44 via the inner lead 77 and the peripheral-face lead 78, such an embodiment is not limiting. For example, as in a sensor element 401 according to a modification illustrated in FIG. 8, the element body 102 may be replaced with an element body 402, and the inner lead 77 and the peripheral-face lead 78 may be replaced with an inner lead 477 and a through-hole conductor 478. The element body 402 of the sensor element 401 has a through-hole 402h. The through-hole 402h has an opening provided at the first face 102a of the element body 402 and extends through the spacer layer 5 and the second solid electrolyte layer 6 in the layered direction (up-down direction). The inner lead 477 is disposed inside the element body 402, specifically, between the first solid electrolyte layer 4 and the spacer layer 5, as with the inner lead 77 of the sensor element 101. At least a part of the inner lead 477 is enclosed at the outer periphery thereof by a lead insulation layer (not illustrated), as with the inner lead 77. The through-hole conductor 478 is disposed in the through-hole 402h with an insulating layer 479 interposed therebetween. The lower end of the through-hole conductor 478 is connected to the inner lead 477. The entirety of the upper end face of the through-hole conductor 478 is covered by and connected to the connector electrode 75a in such a manner as not to be exposed to the outside of the sensor element 401. In such a case, the connector electrode 75a corresponds to the outer conducting section according to the present invention. The connector electrode 75a is the part of the conducting section 74 that contributes to the less likeliness of the gas reaching the measurement electrode 44. Therefore, the connector electrode 75a may be provided as the dense segment according to the present invention, as with the peripheral-face lead 78 of the sensor element 101.

While the connector electrode 75a of the sensor element 401 is constituted of a single layer, the connector electrode 75a may be constituted of two layers or three or more layers. In such a case, the connector electrode 75a can be regarded as either of the peripheral-face leads 278 and 378 of the sensor elements 201 and 301 according to the modifications illustrated in FIGS. 6 and 7.

In each of the above sensor elements 101, 201, 301, and 401, a part of the outer conducting section that covers the inner conducting section is provided as the dense segment according to the present invention. The dense segment according to the present invention only needs to include the part of the outer conducting section that covers the inner conducting section and/or at least a part of the inner conducting section. For example, in each of the sensor elements 101, 201, and 301, at least a part of the inner conducting section, i.e., the inner lead 77, may be provided as the dense segment according to the present invention, as with the peripheral-face lead 78 of the sensor element 101. In the sensor element 401, at least a part of the inner conducting section, i.e., the inner lead 477 and the through-hole conductor 478, may be provided as the dense segment according to the present invention, as with the peripheral-face lead 78 of the sensor element 101. If at least a part of the inner conducting section is dense, some kind of gas outside the sensor element 101 (element body 102) is less likely to move toward the measurement electrode 44 through the inside of the inner conducting section. Furthermore, if the dense part of the inner conducting section has high adhesiveness, the gas is less likely to move toward the measurement electrode 44 through a gap between the dense part and the element body 102 as well. Considering the above, if at least a part of the inner conducting section is provided as the dense segment according to the present invention, some kind of gas outside the sensor element 101 (element body 102) is less likely to reach the measurement electrode 44.

While the sensor element 401 includes the through-hole conductor 478 whose upper end is covered by the connector electrode 75a, such an embodiment is not limiting. For example, as in a sensor element 501 according to a modification illustrated in FIG. 9, the connector electrode 75a may be replaced with a connector electrode 575a, and the through-hole conductor 478 may be replaced with a through-hole conductor 578. As with the through-hole conductor 478, the through-hole conductor 578 is disposed in the through-hole 402h with an insulating layer 579 interposed therebetween, and the lower end of the through-hole conductor 578 is connected to the inner lead 477. The connector electrode 575a has a circular hole 575h. At the lower end face of the connector electrode 575a, the outer periphery of the hole 575h is connected to the outer periphery of the upper end of the through-hole conductor 578. Therefore, the upper end of the through-hole conductor 578 excluding the outer periphery is exposed to the outside of the sensor element 501. In such a case, at least a part of the inner conducting section, i.e., the inner lead 477 and the through-hole conductor 578, only needs to be provided as the dense segment according to the present invention, as with the peripheral-face lead 78 of the sensor element 101. For example, a region of the through-hole conductor 578 that includes the part exposed to the outside of the sensor element 501 may be provided as the dense segment according to the present invention. Alternatively, the entirety of the through-hole conductor 578 may be provided as the dense segment according to the present invention. In addition to such parts, the connector electrode 575a may also be provided as the dense segment according to the present invention, as with the peripheral-face lead 78. Thus, some kind of gas outside the sensor element 501 (element body 102) is less likely to pass through the connector electrode 575a, and to reach the periphery of the through-hole conductor 578, specifically, to enter a gap between the through-hole conductor 578 and the insulating layer 579 or a gap between the insulating layer 579 and the through-hole 402h.

In the above embodiment, the conducting section 74 provided for the measurement electrode 44 has been described. Another conducting sections 74 provided for any of the inner pump electrode 22, the auxiliary pump electrode 51, and the reference electrode 42 may be configured the same as above. For example, as with the case where the conducting section 74 provided for the measurement electrode 44 includes the connector electrode 75a and the lead 76 including the inner lead 77 and the peripheral-face lead 78, if the conducting section 74 provided for the inner pump electrode 22 includes a connector electrode 75h and a lead including an inner lead and a peripheral-face lead, the peripheral-face lead may be provided as the dense segment according to the present invention, as with the peripheral-face lead 78. In addition to or in replacement of such a peripheral-face lead, at least a part of the inner lead may be provided as the dense segment according to the present invention.

While the oxygen-concentration adjustment chamber according to the above embodiment includes the first internal cavity 20 and the second internal cavity 40, such an embodiment is not limiting. For example, the oxygen-concentration adjustment chamber may further include another internal cavity, or one of the first internal cavity 20 and the second internal cavity 40 may be omitted. Likewise, while the adjustment pump cell according to the above embodiment includes the main pump cell 21 and the auxiliary pump cell 50, such an embodiment is not limiting. For example, the adjustment pump cell may further include another pump cell, or one of the main pump cell 21 and the auxiliary pump cell 50 may be omitted. For example, if the oxygen concentration in the measurement-object gas can be satisfactorily reduced by using the main pump cell 21 alone, the auxiliary pump cell 50 may be omitted. If the auxiliary pump cell 50 is omitted, the controller 96 only needs to perform the main-pump control process as the adjustment-pump control process. Furthermore, in the main-pump control process, the setting of the target value V0* based on the pump current Ip1 described above may be omitted. Specifically, if a predetermined target value V0* is preliminarily stored in the storage unit 98, the controller 96 only needs to control the main pump cell 21 by performing feedback control on the voltage Vp0 of the variable power source 24 such that the voltage V0 becomes the target value V0*.

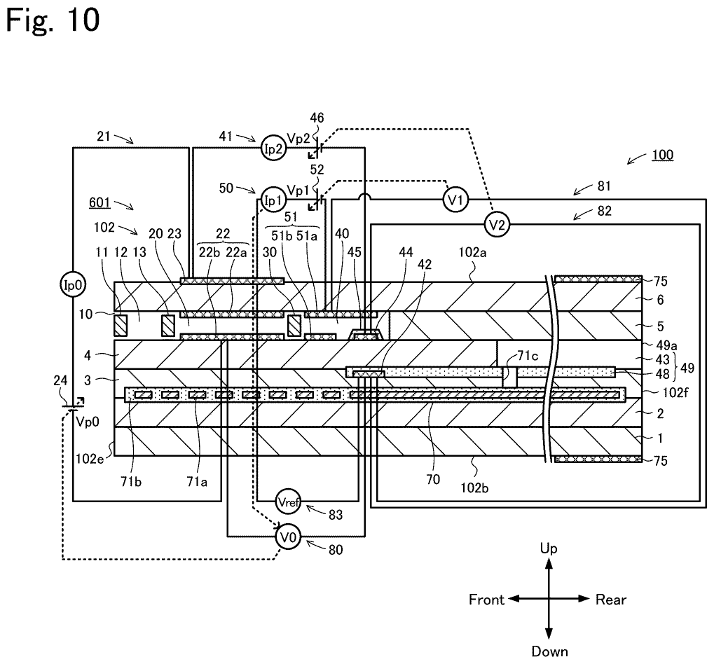

While the sensor element 101 of the gas sensor 100 according to the above embodiment has the first internal cavity 20, the second internal cavity 40, and the third internal cavity 61, such an embodiment is not limiting. For example, the third internal cavity 61 may be omitted, as in a sensor element 601 according to a modification illustrated in FIG. 10. In the sensor element 601 according to the modification illustrated in FIG. 10, the gas inlet 10, the first diffusion control section 11, the buffer space 12, the second diffusion control section 13, the first internal cavity 20, the third diffusion control section 30, and the second internal cavity 40 are provided next to one another and communicate with one another in that order between the lower surface of the second solid electrolyte layer 6 and the upper surface of the first solid electrolyte layer 4. Furthermore, the measurement electrode 44 is disposed on the upper surface of the first solid electrolyte layer 4 in the second internal cavity 40. The measurement electrode 44 is covered by a fourth diffusion control section 45. The fourth diffusion control section 45 is a film formed of a ceramic porous body composed of alumina (Al₂O₃) or the like. Similar to the fourth diffusion control section 60 according to the above embodiment, the fourth diffusion control section 45 has a role of limiting the amount of NOx flowing to the measurement electrode 44. Moreover, the fourth diffusion control section 45 also functions as a protective film for the measurement electrode 44. The ceiling electrode portion 51a of the auxiliary pump electrode 51 extends up to a position straightly above the measurement electrode 44. As with the case of the above embodiment, the sensor element 601 having such a configuration is capable of detecting the NOx concentration by using the measurement pump cell 41. In the sensor element 601 illustrated in FIG. 10, the space around of the measurement electrode 44 functions as a measurement chamber. Specifically, the space around the measurement electrode 44 has a role similar to the role of the third internal cavity 61.

The outer pump electrode 23 according to the above embodiment has a role as an electrode (also referred to as “outer main pump electrode”) paired with the inner pump electrode 22 in the main pump cell 21, a role as an electrode (also referred to as “outer auxiliary pump electrode”) paired with the auxiliary pump electrode 51 in the auxiliary pump cell 50, and a role as an electrode (also referred to as “outer measurement electrode”) paired with the measurement electrode 44 in the measurement pump cell 41. However, such an embodiment is not limiting. Any one or more of the outer main pump electrode, the outer auxiliary pump electrode, and the outer measurement electrode may be disposed separately from the outer pump electrode 23 and outside the element body 102 in such a manner as to come into contact with the measurement-object gas.

While the sensor element 101 according to the above embodiment is configured to detect the concentration of NOx in the measurement-object gas, such an embodiment is not limiting as long as the sensor element is configured to detect the concentration of any specific gas in the measurement-object gas. For example, the concentration of any oxide other than NOx may be defined as the specific gas concentration. If the specific gas is an oxide, oxygen is produced when the specific gas itself is reduced in the third internal cavity 61, as with the case of the above embodiment. Therefore, the measurement pump cell 41 can detect the specific gas concentration by acquiring a value (for example, the pump current Ip2) detected in correspondence with the oxygen. Alternatively, the specific gas may be a non-oxide, such as ammonia. If the specific gas is a non-oxide, the specific gas is converted into an oxide (e.g., into NO in the case of ammonia), so that oxygen is produced when the converted gas is reduced in the third internal cavity 61. Thus, the measurement pump cell 41 can acquire a value (e.g., the pump current Ip2) detected in correspondence with this oxygen and thus detect the specific gas concentration. For example, since the inner pump electrode 22 in the first internal cavity 20 functions as a catalyst, the ammonia is converted into NO in the first internal cavity 20.

While the element body 102 of the sensor element 101 according to the above embodiment is a layered body including a plurality of solid electrolyte layers (i.e., the layers 1 to 6), such an embodiment is not limiting. The element body 102 only needs to include at least one oxygen-ion-conductive solid electrolyte layer. For example, the layers 1 to 5 other than the second solid electrolyte layer 6 in FIG. 2 may be layers (e.g., layers composed of alumina) composed of a material other than that of solid electrolyte layers. In such a case, the electrodes in the sensor element 101 may be disposed on the second solid electrolyte layer 6. For example, the measurement electrode 44 in FIG. 2 may be disposed on the lower surface of the second solid electrolyte layer 6. Moreover, the reference-gas introduction space 43 may be provided in the spacer layer 5 instead of being provided in the first solid electrolyte layer 4, and the reference-gas introduction layer 48 may be disposed between the second solid electrolyte layer 6 and the spacer layer 5 instead of being disposed between the first solid electrolyte layer 4 and the third substrate layer 3, and the reference electrode 42 may be disposed rearward of the third internal cavity 61 and on the lower surface of the second solid electrolyte layer 6. If the layered body includes any layer other than solid electrolyte layers, the dense segment according to the present invention may be in contact with the solid electrolyte layers as with the case of the peripheral-face lead 78 according to the above embodiment, or may be in contact with a layer (for example, a layer composed of alumina) composed of a material other than the solid electrolyte layer.

In the above embodiment, the controller 96 sets (i.e., performs feedback control on) the target value V0* of the voltage V0 based on the pump current Ip1 so as to set the pump current Ip1 to the target value Ip1*, and performs feedback control on the pump voltage Vp0 so as to set the voltage V0 to the target value V0*, but may perform another type of control. For example, the controller 96 may perform feedback control on the pump voltage Vp0 based on the pump current Ip1 so as to set the pump current Ip1 to the target value Ip1*. Specifically, the controller 96 may omit the acquisition of the voltage V0 from the main-pump-control oxygen-partial-pressure detection sensor cell 80 and the setting of the target value V0*, and may directly control the pump voltage Vp0 (and by extension the pump current Ip0) based on the pump current Ip1.

While the above embodiment has been described as an embodiment of the gas sensor 100 including any of the sensor elements 101, 201, and so forth, the above embodiment may also be applied to any of the sensor elements 101, 201, and so forth to be included in a gas sensor 100, of course.

### EXAMPLES

Specifically fabricated examples of the sensor element will now be described. Note that the present invention is not limited to the following examples.

### Examples 1 to 8, Comparative Examples 1 to 3

Examples 1 to 8 were obtained by fabricating the sensor element 101 illustrated in FIG. 2 and the gas sensor 100 illustrated in FIG. 1 through the manufacturing method described above. In fabricating the sensor element 101, ceramic green sheets were each obtained by mixing zirconia particles having 4 mol % of yttria added thereto as a stabilizer with an organic binder and an organic solvent, and then molding the mixture by tape molding. The pattern-forming paste for forming the peripheral-face lead 78 was a paste containing noble metal, alumina, and silica. Among Examples 1 to 8, the volumes Vp [vol %], Va [vol %], and Vs [vol %] of noble metal, alumina, silica contained in the peripheral-face lead 78 were varied as summarized in Table 1 below, by changing the ratio of noble metal, alumina, and silica contained in the pattern-forming paste for forming the peripheral-face lead 78. Comparative Examples 1 to 3 were obtained by fabricating a sensor element 101 in which the peripheral-face lead 78 does not contain one of alumina and silica and fabricating a gas sensor 101 including such a sensor element 101, with the volumes Vp [vol %], Va [vol %], and Vs [vol %] of noble metal, alumina, silica in the peripheral-face lead 78 varied as summarized in Table 1 below.

TABLE 1

| | Volumes of | | | | | | |
|---|---|---|---|---|---|---|---|
| | noble | Volumes of | Volumes of | | | | |
| | metal Vp | alumina Va | silica Vs | | Vp/(Va + | | |
| | [vol %] | [vol %] | [vol %] | Va/Vs | Vs) | Denseness | Adhesiveness |
| Example 1 | 95 | 1 | 4 | 0.3 | 19.0 | A | A |
| Example 2 | 87 | 4 | 9 | 0.4 | 6.7 | A | A |
| Example 3 | 80 | 6 | 14 | 0.4 | 4.0 | A | A |
| Example 4 | 60 | 15 | 25 | 0.6 | 1.5 | B | A |
| Example 5 | 72 | 9 | 19 | 0.5 | 2.6 | A | A |
| Example 6 | 80 | 14 | 6 | 2.3 | 4.0 | B | A |
| Example 7 | 80 | 12 | 8 | 1.5 | 4.0 | A | A |
| Example 8 | 80 | 2 | 18 | 0.1 | 4.0 | A | A |
| Comparison example 1 | 89 | 11 | 0 | — | 8.1 | F | — |
| Comparison example 2 | 60 | 40 | 0 | — | 1.5 | F | — |
| Comparison example 3 | 80 | 0 | 20 | 0 | 4.0 | A | F |

### [Evaluation of Denseness]

For each of the sensor elements 101 according to Examples 1 to 8 and Comparative Examples 1 to 3, the porosity Rp [%] of the peripheral-face lead 78 was measured through the above method. If the porosity Rp was lower than 0.5%, the denseness was evaluated to be very high (A). If the porosity Rp was 0.5% or higher but lower than 3%, the denseness was evaluated to be high (B). If the porosity Rp was 3.0% or higher, the denseness was evaluated to be low (F).

### [Evaluation of Adhesiveness]

For each of the sensor elements 101 according to Examples 1 to 8 and Comparative Example 3, a test for measuring the adhesive strength of the peripheral-face lead 78 with respect to the third face 102c of the element body 102 was conducted in accordance with a known Sebastian method (see “Hakumaku Handobukku (Thin Film Handbook)”, Japan Society for the Promotion of Science, 131st Committee (Thin film) [Ohmsha, Ltd.]). Specifically, the sensor element 101 was first cut into pieces of a predetermined size, whereby test pieces each having the peripheral-face lead 78 on the third face 102c thereof were obtained. Subsequently, a stud pin made of copper and having a diameter of 1.3 mm was bonded to the peripheral-face lead 78 of each of the test pieces with epoxy-based adhesive. Then, a force was applied to the stud pin by using an autograph in such a manner as to peel the peripheral-face lead 78 on the test piece from the third face 102c. If the epoxy-based adhesive was broken before the peripheral-face lead 78 peeled from the element body 102 and the stud pin and the peripheral-face lead 78 were separated from each other, the adhesiveness of the peripheral-face lead 78 was evaluated to be high (A). On the other hand, if the peripheral-face lead 78 peeled from the element body 102 before the stud pin and the peripheral-face lead 78 were separated from each other, the adhesiveness of the peripheral-face lead 78 was evaluated to be low (F). As summarized in Table 1, Comparative Examples 1 and 2 were evaluated to have “low (F)” denseness. Therefore, the evaluation of adhesiveness for Comparative Examples 1 and 2 were skipped.

Table 1 summarizes the peripheral-face leads 78 according to Examples 1 to 8 and Comparative Examples 1 to 3 in terms of the volumes Vp [vol %], Va [vol %], and Vs [vol %]; Va/Vs; Vp/(Va+Vs); the results of denseness evaluation; and the results of adhesiveness evaluation.

As can be seen from Table 1, Comparative Examples 1 and 2, each not containing silica, were evaluated to have “low (F)” denseness, whereas Examples 1 to 8 and Comparative Example 3, each containing silica, were evaluated to have “very high (A)” or “high (B)” denseness. Therefore, it is conceivable that containing silica leads to increased denseness. Comparative Example 3, not containing alumina, was evaluated to have “low (F)” adhesiveness, whereas Examples 1 to 8, each containing alumina and silica, were evaluated to have “high (A)” adhesiveness. Therefore, it is conceivable that containing alumina and silica leads to increased adhesiveness.

Among Examples 3 and 6 to 8, which had the same total volume of alumina and silica (Va+Vs=20 [vol %]), Example 6 did not satisfy Va/Vs≤1.5 and was evaluated to have “high (B)” denseness, whereas Examples 3 and 7 to 8 each satisfied Va/Vs≤1.5 and were evaluated to have “very high (A)” denseness. Therefore, it is conceivable that satisfying Va/Vs≤1.5 further increases the denseness of the peripheral-face lead 78.

Examples 1 to 8 each satisfied 1.5≤Vp/(Va+Vs) and were each evaluated to have “very high (A)” or “high (B)” denseness. Among Examples 1 to 5 and 7 to 8, which each satisfied Va/Vs≤1.5, Example 4 did not satisfy 2.6≤Vp/(Va+Vs) and was evaluated to have “high (B)” denseness, whereas Examples 1 to 3, 5, and 7 to 8 each satisfied 2.6≤Vp/(Va+Vs) and were each evaluated to have “very high (A)” denseness. Therefore, it is conceivable that satisfying 2.6≤Vp/(Va+Vs) further increases the denseness of the peripheral-face lead 78.

### Examples 9 to 11

Examples 9 and 10 were obtained by fabricating a sensor element 101 and a gas sensor 100 with the volumes Vp [vol %], Va [vol %], and Vs [vol %] being the same as those of Examples 1 and 2. Furthermore, Example 11 was obtained by fabricating a sensor element 101 and a gas sensor 100, with the ratio of the ingredients of the pattern-forming paste for forming the peripheral-face lead 78 changed such that the peripheral-face lead 78 contained sodium oxide, as summarized in Table 2 below.

TABLE 2

| | | | | | Ratio of the total | |
|---|---|---|---|---|---|---|
| | Volumes of | | | Volumes of | mass of alkali metal | Effect of |
| | noble | Volumes of | Volumes of | sodium | and alkaline-earth | suppressing the |
| | metal Vp | alumina Va | silica Vs | oxide Vn | metal on an oxide | reduction in |
| | [vol %] | [vol %] | [vol %] | [vol %] | basis [wt %] | the conductivity |
| Example 9 | 95 | 1 | 4 | 0 | 0.07 | A |
| Example 10 | 87 | 4 | 9 | 0 | 0.10 | A |
| Example 11 | 83 | 2 | 14 | 1 | 0.12 | B |

### [Measurement of Alkali Metal and Alkaline-Earth Metal]

For each of Examples 9 to 11, the ratio of the total mass of alkali metal and alkaline-earth metal contained in the peripheral-face lead 78 on an oxide basis was measured through the above method. Specifically, the peripheral-face lead 78 was first peeled from the sensor element 101 and was weighed to 0.5 g for use as a measurement sample, and the measurement sample was put into a PTFE container. Subsequently, a sulfuric-acid solution of 7.5 mL with a ratio of water to sulfuric acid of 1:3 was added into the PTFE container. Furthermore, the PTFE container was put into a stainless-steel container, and the stainless-steel container was then sealed with a lid. The stainless-steel container was further put into a thermostatic oven and was kept at 230° C. for 24 hours, excluding the heating-up period and the cooling-down period, whereby the measurement sample was melted into a solution. The solution was added with water, whereby a sample of 50 mL was obtained. The sample was subjected to ICP-AES for qualitative analysis of elements contained in the sample. Using a test Na concentration obtained in the qualitative analysis and a Na calibration curve obtained in advance, the actual Na concentration was obtained. The actual Na concentration thus obtained was subjected to oxide conversion to calculate the ratio of the mass of sodium oxide (Na₂O) contained in the peripheral-face lead 78. The ratio of the mass of alkali metal and alkaline-earth metal, other than Na, on an oxide basis was also calculated in the same manner. Using the results of these calculations, the ratio of the total mass of alkali metal and alkaline-earth metal contained in the peripheral-face lead 78 on an oxide basis was calculated.

### [Evaluation of Conductivity]

For each of the gas sensors 100 according to Examples 9 to 11, another evaluation was conducted to find whether the conductivity of the peripheral-face lead 78 was reduced after a durability test. First, the offset current for the pump current Ip2 before the durability test was examined as follows. Each of the gas sensors 100 according to Examples 9 to 11 was attached to a pipe such that a front end part of the element body 102 of the sensor element 101 protruded into the pipe. A model gas that did not contain NOx, specifically, a model gas containing nitrogen as a base gas with an oxygen concentration of 0% and a NOx concentration of 0 ppm, was prepared as a measurement-object gas and was caused to flow into the pipe. Meanwhile, the heater control process was started. When the heater 71a reaches 800° C., the adjustment-pump control process and the measurement-pump control process were started. A pump current Ip2 generated in this state was measured as the offset current (a pump current Ip2 generated if the measurement-object gas does not contain NOx). Subsequently, a durability test was conducted. Specifically, for each of the gas sensors 100 according to Examples 9 to 11, exhaust gas from a diesel engine was caused to flow as a measurement-object gas into the pipe. Meanwhile, the heater control process, the adjustment-pump control process, and the measurement-pump control process were continued. In this state, the peripheral-face lead 78 had a temperature of 280° C. This state was maintained for 2000 hours. Then, the offset current for the pump current Ip2 obtained after the durability test was measured in the same manner as before the durability test. Subsequently, for each of the gas sensors 100 according to Examples 9 to 11, the variation (difference) in the offset current that was generated from before the durability test until after the durability test was calculated, and the variation was converted into NOx concentration [ppm], which was taken as the amount of offset variation. If the conductivity of the peripheral-face lead 78 is reduced (if the resistance value increases) after the durability test, the pump current Ip2 flowing through the peripheral-face lead 78 changes, even if the NOx concentration is the same. Hence, if the above amount of offset variation was 7 ppm or smaller, the effect of suppressing the reduction in the conductivity of the peripheral-face lead 78 was evaluated to be high (A). If the amount of offset variation was greater than 7 ppm, the effect of suppressing the reduction in the conductivity of the peripheral-face lead 78 was evaluated to be low (B).

Table 2 summarizes the peripheral-face leads 78 according to Examples 9 to 11 in terms of the volumes Vp [vol %], Va [vol %], and Vs [vol %]; the volume Vn [vol %] of sodium oxide; the ratio of the total mass of alkali metal and alkaline-earth metal on an oxide basis; and the results of the evaluation for the effect of suppressing the reduction in the conductivity.

As can be seen from Table 2, in Example 11, the ratio of the total mass of alkali metal and alkaline-earth metal on an oxide basis was higher than 0.10 wt %, and the effect of suppressing the reduction in the conductivity was evaluated to be “low (B)”. On the other hand, in each of Examples 9 and 10, the ratio of the total mass of alkali metal and alkaline-earth metal on an oxide basis satisfied 0.10 wt % or lower, and the effect of suppressing the reduction in the conductivity was evaluated to be “high (A)”. In each of Examples 9 and 10, the amount of alkali metal and/or alkaline-earth metal contained in the peripheral-face lead 78 was small (the ratio of the total mass on an oxide basis was 0.10 wt % or lower). Therefore, it can be said that the probability of reduction in the conductivity of the peripheral-face lead 78 that may be caused by the electromigration of such metals was lowered.

The present application claims priority from Japanese Patent Application No. 2023-198185 filed Nov. 22, 2023, the entire contents of which are incorporated herein by reference.

What is claimed is:

1. A sensor element configured to detect a concentration of a specific gas in a measurement-object gas, the sensor element comprising:
an element body including an oxygen-ion-conductive solid electrolyte layer and having a columnar shape elongate in a longitudinal direction, the element body having a front end and a rear end that are opposite ends in the longitudinal direction and further having a peripheral face that is a surface extending in the longitudinal direction, a part of the element body that is at the front end being allowed to be exposed to the measurement-object gas;
an inner electrode disposed inside the element body; and
a conducting section including an inner conducting section and an outer conducting section, the inner conducting section being disposed inside the element body and being electrically continuous with the inner electrode, the outer conducting section including a connector electrode disposed at a part of the peripheral face that is near the rear end, the outer conducting section further including a segment disposed on the peripheral face and/or a segment disposed on the peripheral face and exposed to an outside of the sensor element, the outer conducting section being electrically continuous with the inner conducting section,
wherein a part of the outer conducting section that covers the inner conducting section and/or at least a part of the inner conducting section is a dense segment that has a dense composition with noble metal, alumina, and silica.

2. The sensor element according to claim 1,
wherein letting volumes of alumina and silica contained in the dense segment be Va [vol %] and Vs [vol %], respectively, the dense segment satisfies Va/Vs≤1.5.

3. The sensor element according to claim 1,
wherein letting volumes of noble metal, alumina, and silica contained in the dense segment be Vp [vol %], Va [vol %], and Vs [vol %], respectively, the dense segment satisfies 1.5≤Vp/(Va+Vs).

4. The sensor element according to claim 3,
wherein the dense segment satisfies 2.6≤Vp/(Va+Vs).

5. The sensor element according to claim 1,
wherein a ratio of a total mass of alkali metal and alkaline-earth metal contained in the dense segment on an oxide basis satisfies 0.1 wt % or smaller.

6. The sensor element according to claim 1,
wherein the inner electrode is a measurement electrode to be used in detecting the concentration of the specific gas.

7. The sensor element according to claim 1,
wherein the element body is a layered body obtained by stacking a plurality of layers including the solid electrolyte layer in a layered direction that is orthogonal to the longitudinal direction,
wherein the layered body has a first face and a second face that are opposite end faces in the layered direction; and a third face and a fourth face that are opposite end faces in a direction orthogonal to the longitudinal direction and to the layered direction, the first to fourth faces each being the peripheral face,
wherein the inner conducting section includes an inner lead that is routed to the third face or the fourth face, and
wherein the part of the outer conducting section that covers the inner conducting section is a peripheral-face lead disposed on the third face or the fourth face and covering the inner lead.

8. The sensor element according to claim 1,
wherein the element body is a layered body obtained by stacking a plurality of layers including the solid electrolyte layer in a layered direction that is orthogonal to the longitudinal direction,
wherein the layered body has a first face and a second face that are opposite end faces in the layered direction and are each the peripheral face,
wherein the element body has a through-hole with an opening provided in a part of the first face or the second face of the peripheral face that is near the rear end, the through-hole extending through one or more of the plurality of layers in the layered direction,
wherein the outer conducting section is the connector electrode, and
wherein the inner conducting section includes a through-hole conductor disposed in the through-hole and covered by the connector electrode.

9. The sensor element according to claim 1,
wherein the element body is a layered body obtained by stacking a plurality of layers including the solid electrolyte layer in a layered direction that is orthogonal to the longitudinal direction,
wherein the layered body has a first face and a second face that are opposite end faces in the layered direction and are each the peripheral face,
wherein the element body has a through-hole with an opening provided in a part of the first face or the second face of the peripheral face that is near the rear end, the through-hole extending through one or more of the plurality of layers in the layered direction,
wherein the inner conducting section includes a through-hole conductor disposed in the through-hole and exposed to the outside at the opening, and
wherein the dense segment includes at least a part of the through-hole conductor.

10. The sensor element according to claim 1 that is a sensor element to be used in a gas sensor, the gas sensor including:
the sensor element;
a case having a cylindrical shape elongate in the longitudinal direction of the sensor element, the case having a second front end and a second rear end that are opposite ends in the longitudinal direction, the sensor element being disposed inside the case; and
a sealing member sealing the second rear end of the case.

11. A gas sensor comprising:
the sensor element according to claim 1;
a case having a cylindrical shape elongate in the longitudinal direction of the sensor element, the case having a second front end and a second rear end that are opposite ends in the longitudinal direction, the case housing the sensor element; and
a sealing member sealing the second rear end of the case.

12. The sensor element according to claim 2,
wherein letting volumes of noble metal, alumina, and silica contained in the dense segment be Vp [vol %], Va [vol %], and Vs [vol %], respectively, the dense segment satisfies 1.5≤Vp/(Va+Vs).

13. The sensor element according to claim 2,
wherein a ratio of a total mass of alkali metal and alkaline-earth metal contained in the dense segment on an oxide basis satisfies 0.1 wt % or smaller.

14. The sensor element according to claim 2,
wherein the inner electrode is a measurement electrode to be used in detecting the concentration of the specific gas.

15. The sensor element according to claim 2,
wherein the element body is a layered body obtained by stacking a plurality of layers including the solid electrolyte layer in a layered direction that is orthogonal to the longitudinal direction,
wherein the layered body has a first face and a second face that are opposite end faces in the layered direction; and a third face and a fourth face that are opposite end faces in a direction orthogonal to the longitudinal direction and to the layered direction, the first to fourth faces each being the peripheral face,
wherein the inner conducting section includes an inner lead that is routed to the third face or the fourth face, and
wherein the part of the outer conducting section that covers the inner conducting section is a peripheral-face lead disposed on the third face or the fourth face and covering the inner lead.

16. The sensor element according to claim 2,
wherein the element body is a layered body obtained by stacking a plurality of layers including the solid electrolyte layer in a layered direction that is orthogonal to the longitudinal direction,
wherein the layered body has a first face and a second face that are opposite end faces in the layered direction and are each the peripheral face,
wherein the element body has a through-hole with an opening provided in a part of the first face or the second face of the peripheral face that is near the rear end, the through-hole extending through one or more of the plurality of layers in the layered direction,
wherein the outer conducting section is the connector electrode, and
wherein the inner conducting section includes a through-hole conductor disposed in the through-hole and covered by the connector electrode.

17. The sensor element according to claim 2,
wherein the element body is a layered body obtained by stacking a plurality of layers including the solid electrolyte layer in a layered direction that is orthogonal to the longitudinal direction,
wherein the layered body has a first face and a second face that are opposite end faces in the layered direction and are each the peripheral face,
wherein the element body has a through-hole with an opening provided in a part of the first face or the second face of the peripheral face that is near the rear end, the through-hole extending through one or more of the plurality of layers in the layered direction,
wherein the inner conducting section includes a through-hole conductor disposed in the through-hole and exposed to the outside at the opening, and
wherein the dense segment includes at least a part of the through-hole conductor.

18. The sensor element according to claim 2 that is a sensor element to be used in a gas sensor, the gas sensor including:
the sensor element;
a case having a cylindrical shape elongate in the longitudinal direction of the sensor element, the case having a second front end and a second rear end that are opposite ends in the longitudinal direction, the sensor element being disposed inside the case; and
a sealing member sealing the second rear end of the case.

19. A gas sensor comprising:
the sensor element according to claim 2;
a case having a cylindrical shape elongate in the longitudinal direction of the sensor element, the case having a second front end and a second rear end that are opposite ends in the longitudinal direction, the case housing the sensor element; and
a sealing member sealing the second rear end of the case.
